(19) United States
(12) Patent Application Publication
(10) Pub. No.: US 2026/0028681 A1
(43) Pub. Date: Jan. 29, 2026

(54) PROBES AND PROBE SEQUENCES FOR THE DETECTION, IDENTIFICATION AND DIFFERENTIATION OF BACTERIA, PATHOGENICITY ELEMENTS, AND ANTIMICROBIAL RESISTANCE (AMR) GENES, AND METHODS OF DESIGNING, MAKING AND USING

(71) Applicant: The Trustees of Columbia University in the City of New York, New York, NY (US)

(72) Inventors: Amit RANJAN, Fort Lee, NJ (US); Cheng GUO, Fort Lee, NJ (US); Thomas BRIESE, White Plains, NY (US); Walter Ian LIPKIN, New York, NY (US)

(21) Appl. No.: 19/341,642

(22) Filed: Sep. 26, 2025

Related U.S. Application Data
(63) Continuation of application No. PCT/US2024/022175, filed on Mar. 29, 2024.
(60) Provisional application No. 63/455,774, filed on Mar. 30, 2023.

(51) Int. Cl. C12Q 1/689; C12Q 1/6874

(52) U.S. Cl. CPC C12Q 1/689; C12Q 1/6874; C12Q 2600/158

(57) ABSTRACT

Described herein is a database of probe sequences and a set of probes that enable the detection, identification and differentiation of bacteria, and one or more of 16S ribosomal RNA pathogenicity elements, and/or antimicrobial resistance (AMR) genes. These sequences or probes have many uses including but not limited to use in a sequence capture platform and other diagnostic assays. The sequences or probes described herein increase the sensitivity of high-throughput sequencing for detection, identification, and differentiation of bacteria, and one or more of 16S ribosomal RNA, pathogenicity elements, and AMR genes.

### CROSS-REFERENCE TO RELATED APPLICATIONS

This application is a continuation of PCT International Application No. PCT/US2024/022175, filed Mar. 29, 2024, which claims the benefit of U.S. Provisional Application No. 63/455,774, filed Mar. 30, 2023, the contents of each of which is hereby incorporated by reference in its entirety.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### BACKGROUND OF THE INVENTION

Early, accurate differential diagnosis of bacterial infections is critical to reducing morbidity, mortality, and health care costs. It can also reduce the inappropriate use of antibiotics. Multiplex PCR methods in common use for differential diagnosis of bacterial infections can identify potential pathogens but do not provide insights into the presence or expression of antimicrobial resistance (AMR) genes. Moreover, culture-based methods require two to several days to identify pathogens and even longer to provide antibiotic susceptibility profiles (Rhee et al., 2017). Accordingly, physicians typically administer broad-spectrum antibiotics pending acquisition of more specific information (Howell and Davis, 2017).

Antibiotic resistance is the ability of bacteria to resist the effects of antibiotics. This occurs when bacteria evolve mechanisms to neutralize the drugs designed to kill them. Antibiotic resistance is a growing public health concern as it can lead to the spread of antibiotic-resistant infections, which are difficult to treat and can be deadly.

No platform currently permits rapid and simultaneous insights into phylogeny and pathogenicity markers needed to enable the early and precise antibiotic treatment that could reduce morbidity, mortality and economic burden. Moreover, there is currently no method to quickly and accurately identify if a bacterial infection is resistant to one or more antibiotics.

Thus, there is a need for a sensitive cost-effective assay for the detection of bacteria, especially in a clinical setting, as well as features associated with pathogenicity and antibiotic resistance.

### BRIEF SUMMARY OF THE INVENTION

Described herein is a database of probe sequences and a set of probes that enable the detection, identification, and/or differentiation of bacteria and/or pathogenicity elements and/or antimicrobial resistance (AMR) genes and/or 16S ribosomal RNA (rRNA). These sequences or probes have many uses including but not limited to use in a sequence capture platform and other diagnostic assays. The sequences or probes described herein increase the sensitivity of high-throughput sequencing for detection, identification, and/or differentiation of bacteria and/or pathogenicity elements and/or AMR genes and/or 16S ribosomal RNA. The current database of probe sequences or set of probes comprises less than one million oligonucleotides.

To enable efficient detection, identification, and/or differentiation of bacteria and/or pathogenicity elements and/or antimicrobial resistance and/or 16S ribosomal RNA, the database of probe sequences and set of probes was designed to target species-specific or clade-specific gene sequences; and/or 16S ribosomal RNA sequences; and/or virulence factor sequences; and/or AMR genes.

Accordingly, disclosed herein is a method of designing and/or making or constructing a database of probe sequences or a set of probes comprising the following steps.

The first step is to obtain sequence information. In some embodiments, sequence information is obtained for:
(i) one or more species-specific or clade-specific marker gene sequences; or
(ii) one or more 16S ribosomal RNA sequences; or
(iii) one or more virulence factor sequences; or
(iv) one or more AMR gene sequences; or
(v) any combination of (i), (ii), (iii) and (iv)

Sequence information is obtained from any public or private database of sequence information of bacteria and/or 16S ribosomal RNA and/or AMR genes and/or virulence factors, including, but not limited to, Metaphlan4, SILVA, CARD (The Comprehensive Antibiotic Resistance Database) and VFDB (Virulence Factor Database). For example, versions of each of these databases are provided in Table 2, however, additional versions, releases, and updates to these or other databases may be used.

In some embodiments, the combined target sequence dataset can contain over 101,000 genetic targets.

The next step of the method is to break the target sequences into fragments to be the basis of the oligonucleotide probes. The probes are designed to be of a length, and spaced at a distance across the target sequences, such that the total number of probe sequences in the database or probes in the probe set corresponds to a desired range or number. For example, the length and spacing of the probes may be configured to result in less than one million probes. In other embodiments, the length and spacing of the probes may be configured to result in about one million probes. In further embodiments, the length and spacing of the probes may be configured to result in over one million probes.

In some embodiments, the probe length is about 5 nucleotides (“nt”) to about 300 nt. In some embodiments, the probe length is about 10 nt to about 280 nt. In some embodiments, the probe length is about 20 nt to about 260 nt. In some embodiments, the probe length is about 30 nt to about 240 nt. In some embodiments, the probe length is about 40 nt to about 220 nt. In some embodiments, the probe length is about 50 nt to about 200 nt. In some embodiments, the probe length is about 60 nt to about 190 nt. In some embodiments, the probe length is about 70 nt to about 180 nt. In some embodiments, the probe length is about 80 nt to about 170 nt. In some embodiments, the probe length is about 90 nt to about 160 nt. In some embodiments, the probe length is about 100 nt to about 150 nt. In some embodiments, the probe length is about 110 nt to about 140 nt. In some embodiments, the probe length is about 115 nt to about 130 nt. In some embodiments, the probe length is about 120 nt.

In some embodiments, the inter-probe spacing is about 20 nt to about 100 nt tiled across the target sequences. In some embodiments, the inter-probe spacing is about 30 nt to about 90 nt tiled across the target sequences. In some embodiments, the inter-probe spacing is about 40 nt to about 80 nt tiled across the target sequences. In some embodiments, the inter-probe spacing is about 50 nt to about 70 nt tiled across the target sequences.

The generated probes can be further clustered for sequence identity to obtain a certain number of probe sequences or probes. In some embodiments, the generated probes are clustered at about 90% to about 99% sequence identity. In some embodiments, the generated probes are clustered at about 92% to about 98% sequence identity. In some embodiments, the generated probes are clustered at about 94% to about 97% sequence identity. In some embodiments, the generated probes are clustered at about 95% to about 97% sequence identity. In some embodiments, the generated probes are clustered at about 96% sequence identity to obtain less than 1 million probes.

Embodiments of the present disclosure also provide automated systems and methods for designing and/or constructing the database of probe sequences and/or set of probes.

In some embodiments, systems, apparatuses, methods, and computer readable media are provided that use bacterial and sequence information along with analytical tools in a design model for designing and/or constructing the database of probe sequences and/or set of probes. For example, in some embodiments, a first analytical tool using the information from species-specific or clade-specific marker genes sequences and/or from 16S ribosomal RNA sequences and/or virulence factor sequences and/or AMR genes and a second analytical tool to fragment the sequences into oligonucleotides with the desired or advantageous parameters for the probes including but not limited to probe length, spacing distance between the probes on the target sequences, and percentage sequence identity.

A further embodiment of the present disclosure is a database of probe sequences and/or a set of probes designed and/or made or constructed using the methods described herein. In one embodiment, the database of probe sequences and/or set of probes comprises less than one million probes. In another embodiment, the dataset of probe sequences and/or set of probes comprises about one million probes. In a further embodiment, the dataset of probe sequences and/or set of probes comprises more than one million probes.

In one embodiment, the probes are oligonucleotide probes. In a further embodiment, the oligonucleotide probes are synthetic. In one embodiment, the set of probes is in the form of an oligonucleotide probe library. In one embodiment, the oligonucleotides can comprise DNA, RNA, linked nucleic acids (LNA), bridged nucleic acids (BNA) and/or peptide nucleic acids (PNA) as well as any nucleic acids that can be derived naturally or synthesized now or in the future. In one embodiment, the set of probes is in the form of a solution. In a further embodiment, the set of probes is in a solid-state form such as a microarray or bead. In a further embodiment, the oligonucleotides are modified by a composition to facilitate binding to a solid state.

A further embodiment is a database comprising information on the probes including but not limited to the length, nucleotide sequence, and/or origin of each oligonucleotide probe. A further embodiment is a computer-readable storage medium with program code comprising information, e.g., a database, comprising information regarding the probes including but not limited to the length, nucleotide sequence, and/or origin of each oligonucleotide probe.

Additionally, the present disclosure provides a method for constructing a sequencing library for the detection, identification, and/or differentiation of bacteria and/or pathogenicity elements and/or AMR genes using the disclosed set of probes.

The present disclosure also provides systems and methods using the database of probe sequences and/or the set of probes for detecting, identifying and/or differentiating bacteria and/or pathogenicity elements and/or AMR genes in a single sample.

The present disclosure also provides for kits.

The present disclosure also provides a bacterial sequence capture platform for the detection, identification, and/or differentiation of bacterially-derived sequences in a sample.

In some embodiments, the platform comprises a plurality of oligonucleotide probes, wherein the plurality comprises at least one oligonucleotide probe which comprises a hybridization portion partially or fully complementary to a portion of a bacterially-derived sequence selected from the group consisting of a bacterial gene sequence, a 16S ribosomal RNA sequence, a pathogenicity element sequence, a virulence factor sequence, and an antimicrobial resistance (AMR) gene sequence.

In some embodiments, the sequences of the hybridization portions of the oligonucleotide probes cluster at about 90-100% sequence identity.

In some embodiments, each hybridization portion of an oligonucleotide probe is about 5-300 nucleotides in length,

In some embodiments, different hybridization portions that each bind a different portion of the same bacterially-derived sequence are tiled across said bacterially-derived sequence and have an inter-probe spacing of about 20-100 nucleotides.

In some embodiments, the plurality of oligonucleotide probes of the platform comprises 100,000 to 1,000,000 oligonucleotide probes, preferably less than about 1,000,000 oligonucleotide probes.

The present disclosure also provides for methods of using the platform and kits comprising the platform.

### BRIEF DESCRIPTION OF THE DRAWINGS

FIGS. 1A-1B show identification of bacterial species (FIG. 1A) and resistance genes (FIG. 1B) in contrived plasma samples using a bacterial sequence capture platform as described herein. The K. pnemoniae strain has AMR genes for carbapenem (KPC), beta-lactamase (Oxa9, SHV), trimethoprim (dfrA), and efflux pumps (LptD, Kpne-KpnG).

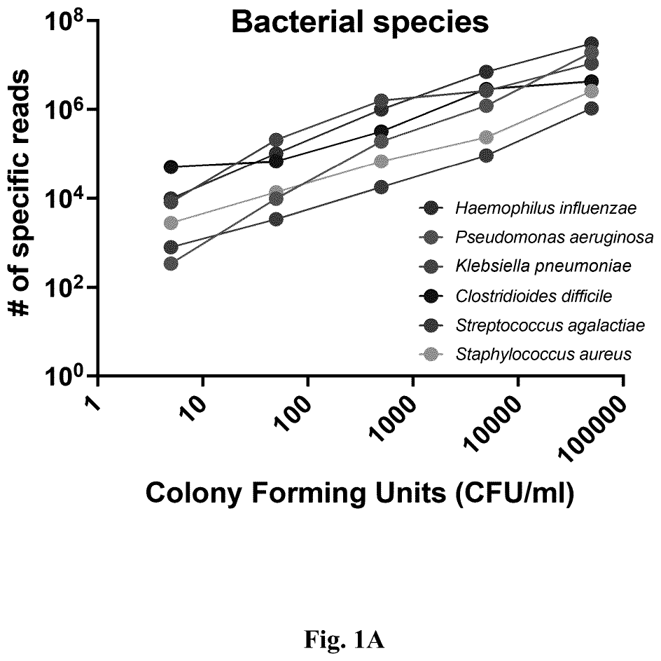

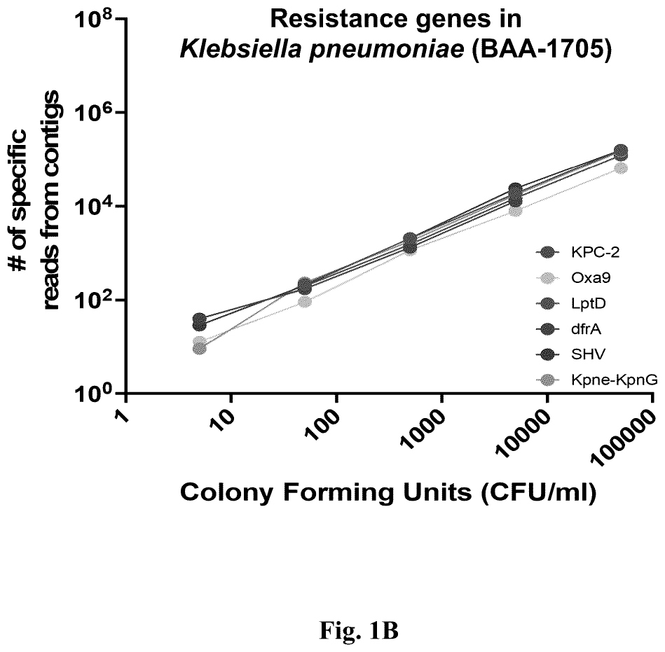

### DETAILED DESCRIPTION OF THE INVENTION

### Molecular Biology

In accordance with the present disclosure, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Definitions

The terms used in this specification generally have their ordinary meanings in the art, within the context of this disclosure and the specific context where each term is used. Certain terms are discussed below, or elsewhere in the specification, to provide additional guidance to the practitioner in describing the disclosed methods and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

As used herein and in the claims, the singular forms “a,” “an,” and “the” include the singular and the plural reference unless the context clearly indicates otherwise. Thus, for example, a reference to “an agent” includes a single agent and a plurality of such agents. Accordingly, it should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

Where a numerical range is provided herein, it is understood that all numerical subsets of that range, and all the individual integers contained therein, are provided as part of the invention. For example, an oligonucleotide probe which is from 100 to 150 nucleotides in length includes the subset of oligonucleotide probes which are 100 to 140 nucleotides in length, the subset of oligonucleotide probes which are 130 to 150 nucleotides in length etc. as well as an oligonucleotide probe which is 100 nucleotides in length, an oligonucleotide probe which is 101 nucleotides in length, an oligonucleotide probe which is 102 nucleotides in length, etc. up to and including an oligonucleotide probe which is 150 nucleotides in length.

As used herein the terms “database of probe sequences” or “database of sequences” and refers to a database comprising information on the probes disclosed herein for the detection, identification, and/or differentiation of bacteria and/or pathogenicity elements and/or AMR genes and/or 16S ribosomal RNA and possibly including the length, nucleotide sequence, and/or origin of each oligonucleotide probe, and computer-readable storage mediums with program code comprising information on the probes disclosed herein for the detection, identification, and/or differentiation of bacteria, and and/or pathogenicity elements, and/or AMR genes and/or 16S ribosomal RNA and possibly including the length, nucleotide sequence, and/or origin of each oligonucleotide probe.

As used herein, the terms “set of probes” or “set of oligonucleotide probes” will be used interchangeably and can refer to the set of probes disclosed herein for the detection, identification, and/or differentiation of bacteria and/or pathogenicity elements and/or AMR genes and/or 16S ribosomal RNA in the form of a collection of synthetic oligonucleotides either in solution or attached to a solid support.

As used herein, the term “oligonucleotide” or “oligonucleotide probe” refers to a nucleic acid that is hybridizable to a genomic DNA molecule, a cDNA molecule, or an mRNA molecule encoding a gene, mRNA, cDNA, or other nucleic acid of interest. The nucleic acids comprised in the oligonucleotides include but are not limited to DNA, RNA, linked nucleic acids (LNA), bridged nucleic acids (BNA) and peptide nucleic acids (PNA). Oligonucleotides can be labeled, e.g., with ³²P-nucleotides or nucleotides to which a label, such as biotin, has been covalently conjugated.

The term “synthetic oligonucleotide” refers to single-stranded DNA or RNA molecules which can be synthesized. In general, these synthetic molecules are designed to have a unique or desired nucleotide sequence, although it is possible to synthesize families of molecules having related sequences and which have different nucleotide compositions at specific positions within the nucleotide sequence. The term synthetic oligonucleotide will be used to refer to DNA or RNA molecules having a designed or desired nucleotide sequence.

The term “subject” as used in this application can mean an animal with an immune system such as avians and mammals. Mammals include canines, felines, rodents, bovine, equines, porcines, ovines, and primates. Avians include, but are not limited to, fowls, songbirds, and raptors. Thus, the methods can be used in veterinary medicine, e.g., to treat companion/domestic animals, farm animals, laboratory animals in zoological parks, and animals in the wild, such as bats and rodents. The subject may also be an invertebrate, such as a tick, mosquito or sand fly. The methods are particularly desirable for human medical applications.

The term “patient” as used in this application means a human subject.

The term “detection”, “detect”, “detecting” and the like as used herein means as used herein means to discover the presence or existence of.

The terms “identification”, “identify”, “identifying” and the like as used herein means to recognize a specific bacterium or bacteria and/or gene or genes and/or nucleic acid or nucleic acids in a sample from a subject.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

As used herein, a “nucleic acid”, and “polynucleotide” and “nucleic acid sequence” and “nucleotide sequence” includes a nucleic acid, an oligonucleotide, a nucleotide, a polynucleotide, and any fragment, variant, or derivative thereof. The nucleic acid or polynucleotide may be double-stranded, single-stranded, or triple-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, and xanthine hypoxanthine. As further used herein, the term “cDNA” refers to an isolated DNA polynucleotide or nucleic acid molecule, or any fragment, derivative, or complement thereof. It may be double-stranded, single-stranded, or triple-stranded, it may have originated recombinantly or synthetically, and it may represent coding and/or noncoding 5′ and/or 3′ sequences.

The term “fragment” when used in reference to a nucleotide sequence refers to portions of that nucleotide sequence. The fragments may range in size from 5 nucleotide residues to the entire nucleotide sequence minus one nucleic acid residue.

The term “genome” as used herein, refers to the entirety of an organism's hereditary information that is encoded in its primary DNA or RNA or nucleotide sequence (DNA or RNA as applicable). The genome includes both the genes and the non-coding sequences. For example, the genome may represent a viral genome, a microbial genome or a mammalian genome.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, the terms “complementary” or “complementarity” are used in reference to “polynucleotides” and “oligonucleotides” (which are interchangeable terms that refer to a sequence of nucleotides) related by the base-pairing rules. It may also include mimics of or artificial bases that may not faithfully adhere to the base-pairing rules. For example, the sequence “C-A-G-T,” is complementary to the sequence “G-T-C-A.” In another example, a nucleotide sequence of 5′-CAGT-3′ is complementary to, and is capable of hybridizing to, a nucleotide sequence of 3′-GTCA-5′. Complementarity can be “partial” or “total.” “Partial” complementarity is where one or more nucleic acid bases are not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The term “nucleic acid hybridization” or “hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid).

As used herein the term “hybridization product” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization product may be formed in solution or between one nucleic acid sequence present in solution and another nucleic acid sequence immobilized to a solid support.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences is usually low between such organisms).

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, and GCG (Genetics Computer Group, Program Manual for the GCG Package, Version 7, Madison, Wisconsin).

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

“Amplification” is defined as the production of additional copies of a nucleic acid sequence and is generally carried out either in vivo, or in vitro, i.e. for example using polymerase chain reaction.

As used herein, the term “polymerase chain reaction” (“PCR”) refers to the method disclosed in U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ₃₂P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications. With PCR, it is also possible to amplify a complex mixture (library) of linear DNA molecules, provided they carry suitable universal sequences on either end such that universal PCR primers bind outside of the DNA molecules that are to be amplified.

The terms “next-generation sequencing platform” and “high-throughput sequencing” and “HTS” as used herein, refer to any nucleic acid sequencing device that utilizes massively parallel technology. For example, such a platform may include, but is not limited to, Illumina sequencing platforms.

The term “sequencing library”, as used herein refers to a library of nucleic acids that are compatible with next-generation high throughput sequencers.

The term “bacterially-derived sequence” as used herein refers to a sequence which is typically associated with bacteria. For example, the sequence may be a sequence present in a bacterial genome, or a sequence from a plasmid, virus, or bacteriophage known to be harbored by one or more bacterial species.

The term “hybridization portion” as used herein in the context of an oligonucleotide probe of a bacterial sequence capture platform refers to a portion of a oligonucleotide probe that is partially or fully complementary to a bacterially-derived sequence. For example, the hybridization portion of an oligonucleotide probe may hybridize to a target bacterially-derived sequence on a tested nucleotide molecule when the oligonucleotide probe is exposed to a sample containing the tested nucleotide molecule.

The term “pathogenicity element sequence” is a nucleotide sequence associated with increasing the pathogenicity (i.e., the capacity to cause disease) of an organism.

The term “virulence factor sequence” refers to a nucleotide sequence which encodes a product that enables a microorganism to establish itself on or within a host of a particular species and enhance its potential to cause disease. For example, virulence factors include, but are not limited to, bacterial toxins, cell surface proteins that mediate bacterial attachment, cell surface carbohydrates, proteins that protect a bacterium, and hydrolytic enzymes that may contribute to bacterial pathogenicity.

The term “environmental sample” as used herein refers to a sample obtained from any non-biological media or material(s), including but not limited to, air, soil, water, and swabs of inanimate surfaces. Environmental samples contrast with biological samples, which typically derive from an organism. Examples of biological samples include, but are not limited to, bodily fluids, cells, tissue samples, and swabs of a surface or cavity of a biological organism.

The following embodiments and examples (including details thereof) are set forth to aid in an understanding of the subject matter of this disclosure but are not intended to, and should not be construed to, limit in any way the invention that is claimed.

### Database of Probe Sequences and Set of Probes

Described herein is a database of probe sequences and a set of probes that enable the detection, identification and/or differentiation of bacteria, as well as pathogenicity elements, and/or antimicrobial resistance (AMR) genes and/or 16S ribosomal RNA. These sequences or probes have many uses including but not limited to use in a sequence capture platform and other diagnostic assays. The sequences or probes described herein increase the sensitivity of high-throughput sequencing for detection, identification, and/or differentiation of bacteria and/or pathogenicity elements and/or AMR genes and/or 16S ribosomal RNA.

The database of probe sequences or set of probes is comprised of oligonucleotides that are distributed across informative regions of bacteria. For example, the database of probe sequences or set of probes may comprise about one million or fewer oligonucleotides. To enable efficient detection, identification, and/or differentiation of bacteria, and/or virulence elements and/or antimicrobial resistance and/or 16S ribosomal RNA, the database of probe sequences and set of probes can be designed to target four major components: 1. Sequence-specific or clade-specific marker genes sequences extracted, for example one or more such sequences from the Metaphlan4 database; or 2. 16S ribosomal RNA sequences, for example one or more such sequences extracted from SILVA database for a total of 1333 bacterial species (see Table 1); or 3. Virulence factors genetic sequences in bacterial pathogens, for example one or more such sequences extracted from the VFDB (Virulence Factor Database); or 4. Antibiotic resistance determinants genes, for example one or more such sequences extracted from CARD (The Comprehensive Antibiotic Resistance Database) or any combination of the four. In one embodiment, oligonucleotide probes were designed to bind to regions distributed across the combined target sequence dataset (101,185 genetic fragments=90,776 for 894 species from Metaphlan4+1325 species from SILVA 16S+4750 AMR+4334 VFDB) (Table 2). The generated probes were further clustered for sequence identity, which resulted in 988,786 probes.

The database of probe sequences and set of probes disclosed and described herein are more targeted than prior known databases and sets of probes and can identify the bacteria in any given sample by targeting species-specific or clade-specific marker sequences in bacterial genomes, rather than the entire genome of bacteria.

Other differences from prior known databases and probe sets are a longer uniform probe size and smaller number of probes (e.g., one million or less). There is also no adjustment of length for Tm of the probes. Additionally, the probe set may include 16S ribosomal RNA sequences and/or AMR genes and/or virulence factor genes. After all of the sequences were obtained, they were clustered for sequence identity to reduce or eliminate redundancy. This resulted in a database of probe sequences and set of probes that was less redundant than previous sets. Additionally, over 1,300 different bacteria can be identified using the disclosed database of probe sequences or set of probes (Table 1). The disclosed database of probe sequences or set of probes also leads to more straightforward analysis. For example, the platform of oligonucleotide probes described herein enables detection of bacterially-derived sequences in environmental samples, for example, to determine the prevalence of medically relevant bacteria, pathogenesis elements, virulence factors, and/or AMR sequences in a sample. The disclosed platform or probe set enables a faster, more cost-effective approach to detecting medically relevant bacterially-derived sequences in environmental or clinical samples without sacrificing coverage or accuracy.

The current disclosure includes a method of designing and/or making or constructing a database of probe sequences or set of probes and methods of using the set of probes to construct sequencing libraries suitable for sequencing in any high throughput sequencing technology. The disclosure also includes methods and systems for detecting, identifying and/or differentiating bacteria and/or pathogenic elements and/or AMR genes and/or 16S ribosomal RNA in a single sample, of any origin, using the database of probe sequences or set of probes. The database of probe sequences or set of probes enables detection of bacterial sequences in any complex sample background, including those found in clinical specimens and the presence of features associated with pathogenicity and/or antimicrobial resistance.

The present disclosure includes a method of designing and/or constructing a database of probe sequences or set of probes for the detection, identification, and/or differentiation of bacteria and/or pathogenicity elements and/or AMR genes and/or 16S ribosomal RNA. Accordingly, the method may include the following steps.

The first step is to obtain sequence information including species-specific or clade-specific marker gene of bacteria, or 16S ribosomal RNA sequences, or AMR genes, or virulence factors, or a combination of any of the four.

Sequence information is obtained from any public or private database of sequence information of bacteria, 16S ribosomal RNA sequences, AMR genes and/or virulence factors, including, but not limited, to Metaphlan4, SILVA, CARD and VFDB. Any version of these databases, including but not limited to those exemplified in Table 2, as well as future updates, may be used.

The next step of the method is to break the sequences into fragments to be the basis of the oligonucleotide probes. In the current embodiment, the probes are spaced at a distance across the target sequences, such that the total number of probe sequences in the database or probes in the probe set is about one million or less and cover all target sequences.

In some embodiments, the probe length is about 5 nt to about 300 nt. In some embodiments, the probe length is about 10 nt to about 280 nt. In some embodiments, the probe length is about 20 nt to about 260 nt. In some embodiments, the probe length is about 30 nt to about 240 nt. In some embodiments, the probe length is about 40 nt to about 220 nt. In some embodiments, the probe length is about 50 nt to about 200 nt. In some embodiments, the probe length is about 60 nt to about 190 nt. In some embodiments, the probe length is about 70 nt to about 180 nt. In some embodiments, the probe length is about 80 nt to about 170 nt. In some embodiments, the probe length is about 90 nt to about 160 nt. In some embodiments, the probe length is about 100 nt to about 150 nt. In some embodiments, the probe length is about 110 nt to about 140 nt. In some embodiments, the probe length is about 115 nt to about 130 nt. In some embodiments, the probe length is about 120 nt.

In some embodiments, the inter-probe spacing is about 20 nt to about 100 nt tiled across the target sequences. In some embodiments, the inter-probe spacing is about 30 nt to about 90 nt tiled across the target sequences. In some embodiments, the inter-probe spacing is about 40 nt to about 80 nt tiled across the target sequences. In some embodiments, the inter-probe spacing is about 50 nt to about 70 nt tiled across the target sequences. In some embodiments, the inter-probe spacing is about 60 nt tiled across the target sequences.

The generated probes can be further clustered for sequence identity to obtain a certain number of probe sequences or probes. In some embodiments, the generated probes are clustered at about 90% to about 99% sequence identity. In some embodiments, the generated probes are clustered at about 92% to about 98% sequence identity. In some embodiments, the generated probes are clustered at about 94% to about 97% sequence identity. In some embodiments, the generated probes are clustered at about 95% to about 97% sequence identity.

In some embodiments, the generated probes are clustered at about 96% sequence identity which resulted in less than one million (988,786) probes.

Specifically, oligonucleotides are selected to bind to regions distributed across the combined target sequence dataset, which in the current embodiment was 101,185 genetic targets, corresponding to 90,776 genes in 894 species from Metaphlan4, 1325 rRNA sequences from SILVA 16S, 4750 AMR genes from CARD, and 4334 virulence factor sequences from VFDB.

Any bacterially-derived sequences desired to be targeted, preferably sequences which are relevant to pathogenesis and/or virulence or are otherwise medically relevant, may be used to generate oligonucleotides probes for use in any one of the probe sets or bacterial sequence capture platforms described herein, or used to generate a database of probe sequences. For example, sequence information of desired targets may be obtained from any public or private database of sequence information of bacteria and/or 16S ribosomal RNA and/or AMR genes and/or virulence factors, including, but not limited to, Metaphlan4, SILVA, CARD, and VFDB. For example, versions of each of these databases are provided in Table 2, however, additional versions, releases, and updates to these or other databases may be used.

Metaphlan4 (Metagenomic Phylogenetic Analysis 4) is a computational tool for species-level microbial profiling. See huttenhower.sph.harvard.edu/metaphlan and Aitor Blanco-Miguez et al. (2022) “Extending and improving metagenomic taxonomic profiling with uncharacterized species with MetaPhlAn 4”, bioRxiv preprint doi.org/10.1101/2022.08.22.504593, the contents of both of which are incorporated herein by reference.

SILVA is a high-quality ribosomal RNA database. Release information of the SILVA SSU and LSU databases 138.1 as of Aug. 27, 2020 is available at www.arb-silva.de/documentation/release-1381/, the content of which is incorporated herein by reference.

CARD (The Comprehensive Antibiotic Resistance Database) is a bioinformatic database of resistance genes, their products and associated phenotypes. See card.mcmaster.ca/home and Alcock B P et al. “CARD 2023: expanded curation, support for machine learning, and resistome prediction at the Comprehensive Antibiotic Resistance Database.” Nucleic Acids Res. 2023 Jan. 6; 51 (D1): D690-D699, the contents of both of which are incorporated herein by reference.

VFDB (Virulence Factor Database) is an integrated and comprehensive online resource for curating information about virulence factors of bacterial pathogens. See mgc.ac.cn/VFs/main.htm and Liu B et al. “VFDB 2022: a general classification scheme for bacterial virulence factors.” Nucleic Acids Res. 2022 Jan. 7; 50 (D1): D912-D917, the contents of both of which are incorporated herein by reference.

TABLE 1
Medically Important Bacterial Species

Abiotrophia defectiva
Acetobacter nitrogenifigens
Achromobacter denitrificans
Achromobacter insolitus
Achromobacter piechaudii
Achromobacter ruhlandii
Achromobacter xylosoxidans
Acidaminococcus fermentans
Acidaminococcus intestini
Acidovorax citrulli
Acinetobacter baumannii
Acinetobacter bereziniae
Acinetobacter calcoaceticus
Acinetobacter haemolyticus
Acinetobacter johnsonii
Acinetobacter junii
Acinetobacter lwoffii
Acinetobacter parvus
Acinetobacter pittii
Acinetobacter radioresistens
Acinetobacter schindleri
Acinetobacter seifertii
Acinetobacter soli
Acinetobacter ursingii
Actinobacillus hominis
Actinobacillus suis
Actinobacillus ureae
Actinobaculum massiliense
Actinomadura madurae
Actinomadura pelletieri
Actinomyces cardiffensis
Actinomyces georgiae
Actinomyces gerencseriae
Actinomyces graevenitzii
Actinomyces hongkongensis
Actinomyces israelii
Actinomyces massiliensis
Actinomyces meyeri
Actinomyces naeslundii
Actinomyces neuii
Actinomyces neuii anitratus
Actinomyces neuii neuii
Actinomyces oris
Actinomyces radicidentis
Actinomyces radingae
Actinomyces timonensis
Actinomyces turicensis
Actinomyces urogenitalis
Actinomyces viscosus
Advenella incenata
Aerococcus christensenii
Aerococcus sanguinicola
Aerococcus urinae
Aerococcus urinaeequi
Aerococcus urinaehominis
Aerococcus viridans
Aeromonas bestiarum
Aeromonas caviae
Aeromonas enteropelogenes
Aeromonas hydrophila
Aeromonas salmonicida
Aeromonas schubertii
Aeromonas veronii
Afipia birgiae
Afipia broomeae
Afipia clevelandensis
Afipia felis
Aggregatibacter actinomycetemcomitans
Aggregatibacter aphrophilus
Aggregatibacter segnis
Agrobacterium tumefaciens
Alcaligenes faecalis
Alistipes finegoldii
Alistipes onderdonkii
Alistipes putredinis
Alistipes shahii
Alloiococcus otitis
Alloprevotella tannerae
Alloscardovia omnicolens
Alysiella crassa
Amycolatopsis palatopharyngis
Anaerobiospirillum succiniciproducens
Anaerococcus hydrogenalis
Anaerococcus lactolyticus
Anaerococcus octavius
Anaerococcus prevotii
Anaerococcus tetradius
Anaerococcus vaginalis
Anaeroglobus geminatus
Anaerostipes caccae
Anaplasma phagocytophilum
Arcanobacterium haemolyticum
Arcobacter butzleri
Arcobacter cryaerophilus
Arcobacter skirrowii
Arthrobacter oxydans
Arthrobacter scleromae
Arthrobacter woluwensis
Atopobium parvulum
Atopobium rimae
Atopobium vaginae
Aureimonas altamirensis
Bacillus anthracis
Bacillus cereus
Bacillus circulans
Bacillus coagulans
Bacillus glycinifermentans
Bacillus licheniformis
Bacillus megaterium
Bacillus mycoides
Bacillus paralicheniformis
Bacillus paucivorans
Bacillus pumilus
Bacillus safensis
Bacillus sphaericus
Bacillus subtilis
Bacillus thuringiensis
Bacteroides caccae
Bacteroides distasonis
Bacteroides eggerthii
Bacteroides faecis
Bacteroides finegoldii
Bacteroides fragilis
Bacteroides massiliensis
Bacteroides merdae
Bacteroides nordii
Bacteroides ovatus
Bacteroides pyogenes
Bacteroides stercoris
Bacteroides thetaiotaomicron
Bacteroides uniformis
Bacteroides vulgatus
Balneatrix alpica
Bartonella alsatica
Bartonella ancashensis
Bartonella bacilliformis
Bartonella birtlesii
Bartonella bovis
Bartonella clarridgeiae
Bartonella doshiae
Bartonella elizabethae
Bartonella grahamii
Bartonella henselae
Bartonella koehlerae
Bartonella quintana
Bartonella rattaustraliani
Bartonella rochalimae
Bartonella schoenbuchensis
Bartonella taylorii
Bartonella tribocorum
Bartonella vinsonii
Bartonella vinsonii subsp. Vinsonii¬†
Bergeyella zoohelcum
Bifidobacterium adolescentis
Bifidobacterium angulatum
Bifidobacterium animalis
Bifidobacterium bifidum
Bifidobacterium breve
Bifidobacterium dentium
Bifidobacterium infantis
Bifidobacterium longum
Bifidobacterium pseudocatenulatum
Bifidobacterium psychraerophilum
Bifidobacterium scardovii
Bilophila wadsworthia
Bordetella avium
Bordetella bronchialis
Bordetella bronchiseptica
Bordetella flabilis
Bordetella hinzii
Bordetella holmesii
Bordetella parapertussis
Bordetella pertussis
Bordetella petrii
Bordetella trematum
Borrelia afzelii
Borrelia crocidurae
Borrelia duttonii
Borrelia garinii
Borrelia hermsii
Borrelia hispanica
Borrelia mayonii
Borrelia miyamotoi
Borrelia parkeri
Borrelia persica
Borrelia recurrentis
Borrelia sinica
Borrelia spielmanii
Borrelia turicatae
Borrelia valaisiana
Borreliella burgdorferi
Bosea massiliensis
Brachyspira aalborgi
Brachyspira pilosicoli
Brevibacillus brevis
Brevibacillus centrosporus
Brevibacillus laterosporus
Brevibacillus parabrevis
Brevibacterium casei
Brevundimonas diminuta
Brevundimonas vesicularis
Brucella abortus
Brucella canis
Brucella inopinata
Brucella melitensis
Brucella suis
Budvicia aquatica
Bulleidia extructa
Burkholderia ambifaria
Burkholderia anthina
Burkholderia cenocepacia
Burkholderia cepacia
Burkholderia dolosa
Burkholderia fungorum
Burkholderia gladioli
Burkholderia glumae
Burkholderia mallei
Burkholderia multivorans
Burkholderia oklahomensis
Burkholderia pseudomallei
Burkholderia pyrrocinia
Burkholderia stabilis
Burkholderia thailandensis
Burkholderia vietnamiensis
Burkholderiales bacterium
Burkholderiales bacterium 8X
Burkholderiales bacterium C2
Burkholderiales bacterium GJ E10
Burkholderiales bacterium JOSHI 001
Burkholderiales bacterium LSUCC0115
Buttiauxella agrestis
Buttiauxella brennerae
Buttiauxella ferragutiae
Buttiauxella gaviniae
Butyrivibrio fibrisolvens
Campylobacter coli
Campylobacter concisus
Campylobacter corcagiensis
Campylobacter cuniculorum
Campylobacter curvus
Campylobacter fetus
Campylobacter gracilis
Campylobacter hominis
Campylobacter hyointestinalis
Campylobacter iguaniorum
Campylobacter jejuni
Campylobacter jejuni doylei
Campylobacter jejuni jejuni
Campylobacter lari
Campylobacter mucosalis
Campylobacter rectus
Campylobacter showae
Campylobacter sputorum
Campylobacter upsaliensis
Campylobacter ureolyticus
Candidatus Bartonella
Capnocytophaga canimorsus
Capnocytophaga cynodegmi
Capnocytophaga gingivalis
Capnocytophaga granulosa
Capnocytophaga ochracea
Capnocytophaga sputigena
Cardiobacterium hominis
Cardiobacterium valvarum
Catabacter hongkongensis
Catonella morbi
Cedecea davisae
Cedecea lapagei
Cedecea neteri
Cellulomonas flavigena
Cellulomonas hominis
Cellulosimicrobium cellulans
Cellulosimicrobium funkei
Centipeda periodontii
Chlamydia pneumonia
Chlamydia pneumoniae
Chlamydia psittaci
Chlamydia trachomatis
Chromobacterium haemolyticum
Chromobacterium violaceum
Chryseobacterium
Chryseobacterium gleum
Chryseobacterium indologenes
Citrobacter amalonaticus
Citrobacter braakii
Citrobacter farmeri
Citrobacter freundii
Citrobacter koseri
Citrobacter murliniae
Citrobacter rodentium
Citrobacter sedlakii
Citrobacter werkmanii
Citrobacter youngae
Clostridium argentinense
Clostridium baratii
Clostridium beijerinckii
Clostridium bifermentans
Clostridium bolteae
Clostridium botulinum
Clostridium butyricum
Clostridium cadaveris
Clostridium carnis
Clostridium celatum
Clostridium cochlearium
Clostridium cocleatum
Clostridium difficile
Clostridium fallax
Clostridium ghonii
Clostridium haemolyticum
Clostridium hylemonae
Clostridium indolis
Clostridium innocuum
Clostridium leptum
Clostridium neonatale
Clostridium novyi
Clostridium paraputrificum
Clostridium perfringens
Clostridium piliforme
Clostridium ramosum
Clostridium septicum
Clostridium sordellii
Clostridium sphenoides
Clostridium spiroforme
Clostridium sporogenes
Clostridium subterminale
Clostridium symbiosum
Clostridium tertium
Clostridium tetani
Collinsella aerofaciens
Comamonas kerstersii
Comamonas terrigena
Comamonas testosteroni
Corynebacterium accolens
Corynebacterium afermentans
Corynebacterium amycolatum
Corynebacterium argentoratense
Corynebacterium aurimucosum
Corynebacterium auris
Corynebacterium bovis
Corynebacterium confusum
Corynebacterium coyleae
Corynebacterium diphtheriae
Corynebacterium durum
Corynebacterium falsenii
Corynebacterium freiburgense
Corynebacterium freneyi
Corynebacterium glucuronolyticum
Corynebacterium halotolerans
Corynebacterium imitans
Corynebacterium jeikeium
Corynebacterium kroppenstedtii
Corynebacterium kutscheri
Corynebacterium lipophiloflavum
Corynebacterium macginleyi
Corynebacterium massiliense
Corynebacterium matruchotii
Corynebacterium minutissimum
Corynebacterium mucifaciens
Corynebacterium mycetoides
Corynebacterium pilosum
Corynebacterium propinquum
Corynebacterium pseudodiphtheriticum
Corynebacterium pseudotuberculosis
Corynebacterium renale
Corynebacterium resistens
Corynebacterium riegelii
Corynebacterium sanguinis
Corynebacterium simulans
Corynebacterium singulare
Corynebacterium stationis
Corynebacterium striatum
Corynebacterium sundsvallense
Corynebacterium thomssenii
Corynebacterium timonense
Corynebacterium tuberculostearicum
Corynebacterium tuscaniense
Corynebacterium ulcerans
Corynebacterium urealyticum
Corynebacterium ureicelerivorans
Corynebacterium vitaeruminis
Corynebacterium xerosis
Coxiella burnetii
Cronobacter condimenti
Cronobacter dublinensis
Cronobacter malonaticus
Cronobacter sakazakii
Cronobacter turicensis
Cronobacter universalis
Cryptobacterium curtum
Cupriavidus gilardii
Cupriavidus metallidurans
Cupriavidus pauculus
Cupriavidus taiwanensis
Delftia acidovorans
Dermabacter hominis
Dermacoccus abyssi
Dermacoccus nishinomiyaensis
Dermatophilus congolensis
Desulfomicrobium orale
Desulfovibrio desulfuricans
Desulfovibrio fairfieldensis
Desulfovibrio vulgaris
Dialister invisus
Dialister micraerophilus
Dialister pneumosintes
Dialister propionicifaciens
Dichelobacter nodosus
Dielma fastidiosa
Dietzia maris
Dolosicoccus paucivorans
Dolosigranulum pigrum
Dysgonomonas capnocytophagoides
Dysgonomonas gadei
Dysgonomonas hofstadii
Dysgonomonas mossii
Edwardsiella hoshinae
Edwardsiella ictaluri
Edwardsiella tarda
Eggerthella hongkongensis
Eggerthella lenta
Eggerthella sinensis
Ehrlichia canis
Ehrlichia chaffeensis
Ehrlichia muris
Eikenella corrodens
Elizabethkingia anophelis
Elizabethkingia meningoseptica
Elizabethkingia miricola
Empedobacter brevis
Empedobacter falsenii
Enterobacter aerogenes
Enterobacter cancerogenus
Enterobacter cloacae
Enterobacter gergoviae
Enterobacter hormaechei
Enterobacter kobei
Enterobacter ludwigii
Enterobacter mori
Enterobacter sakazakii
Enterococcus asini
Enterococcus avium
Enterococcus casseliflavus
Enterococcus cecorum
Enterococcus columbae
Enterococcus dispar
Enterococcus durans
Enterococcus faecalis
Enterococcus faecium
Enterococcus flavescens
Enterococcus gallinarum
Enterococcus gilvus
Enterococcus haemoperoxidus
Enterococcus hirae
Enterococcus italicus
Enterococcus malodoratus
Enterococcus mundtii
Enterococcus pallens
Enterococcus phoeniculicola
Enterococcus pseudoavium
Enterococcus raffinosus
Enterococcus saccharolyticus
Enterococcus sulfureus
Enterococcus thailandicus
Erwinia billingiae
Erwinia gerundensis
Erysipelatoclostridium ramosum
Erysipelothrix rhusiopathiae
Escherichia albertii
Escherichia coli
Escherichia fergusonii
Eubacterium brachy
Eubacterium infirmum
Eubacterium limosum
Eubacterium minutum
Eubacterium nodatum
Eubacterium rectale
Eubacterium saphenum
Eubacterium sulci
Eubacterium tenue
Eubacterium ventriosum
Eubacterium yurii
Eubacterium yurii mararetiae
Eubacterium yurii schtitka
Eubacterium yurii yurii
Ewingella americana
Exiguobacterium acetylicum
Exiguobacterium aurantiacum
Facklamia hominis
Facklamia ignava
Facklamia languida
Facklamia sourekii
Faecalicoccus pleomorphus
Fenollaria massiliensis
Filifactor alocis
Finegoldia magna
Francisella hispaniensis
Francisella noatunensis
Francisella philomiragia
Francisella tularensis
Franconibacter helveticus
Fusobacterium gonidiaformans
Fusobacterium mortiferum
Fusobacterium naviforme
Fusobacterium necrogenes
Fusobacterium necrophorum
Fusobacterium nucleatum
Fusobacterium nucleatum fusiforme
Fusobacterium nucleatum nucleatum
Fusobacterium nucleatum polymorphum
Fusobacterium nucleatum vincentii
Fusobacterium periodonticum
Fusobacterium russii
Fusobacterium ulcerans
Fusobacterium varium
Gardnerella vaginalis
Gemella bergeri
Gemella haemolysans
Gemella morbillorum
Gemella sanguinis
Globicatella sanguinis
Gordonia araii
Gordonia bronchialis
Gordonia otitidis
Gordonia polyisoprenivorans
Gordonia rubripertincta
Gordonia sputi
Gordonia terrae
Gordonibacter pamelaeae
Granulibacter bethesdensis
Granulicatella adiacens
Granulicatella elegans
Grimontia hollisae
Haemophilus aegyptius
Haemophilus ducreyi
Haemophilus haemolyticus
Haemophilus influenzae
Haemophilus parahaemolyticus
Haemophilus parainfluenzae
Haemophilus paraphrohaemolyticus
Haemophilus pittmaniae
Haemophilus quentini
Haemophilus sputorum
Hafnia alvei
Hafnia paralvei
Helcococcus kunzii
Helcococcus sueciensis
Helicobacter bilis
Helicobacter canadensis
Helicobacter canis
Helicobacter cinaedi
Helicobacter felis
Helicobacter fennelliae
Helicobacter heilmannii
Helicobacter magdeburgensis
Helicobacter pullorum
Helicobacter pylori
Helicobacter winghamensis
Holdemania filiformis
Ignatzschineria larvae
Ignavigranum ruoffiae
Inquilinus limosus
Isoptericola variabilis
Janibacter indicus
Janibacter melonis
Johnsonella ignava
Jonesia denitrificans
Kerstersia gyiorum
Kingella denitrificans
Kingella kingae
Kingella oralis
Kingella potus
Klebsiella granulomatis
Klebsiella michiganensis
Klebsiella oxytoca
Klebsiella pneumoniae
Klebsiella pneumoniae ssp. Ozaenae
Klebsiella pneumoniae ssp. Pneumoniae
Klebsiella quasipneumoniae
Klebsiella variicola
Kluyvera ascorbata
Kluyvera cryocrescens
Kluyvera intermedia
Kocuria kristinae
Kocuria palustris
Kocuria rhizophila
Kocuria rosea
Kocuria varians
Kurthia gibsonii
Kurthia huakuii
Kurthia massiliensis
Kytococcus schroeteri
Kytococcus sedentarius
Lactobacillus acidophilus
Lactobacillus antri
Lactobacillus brevis
Lactobacillus casei
Lactobacillus coleohominis
Lactobacillus crispatus
Lactobacillus fermentum
Lactobacillus gasseri
Lactobacillus iners
Lactobacillus jensenii
Lactobacillus paracasei
Lactobacillus paraplantarum
Lactobacillus plantarum
Lactobacillus pontis
Lactobacillus rhamnosus
Lactobacillus saerimneri
Lactobacillus sakei
Lactobacillus salivarius
Lactobacillus ultunensis
Lactobacillus vaginalis
Lactococcus garvieae
Lactococcus lactis
Laribacter hongkongensis
Latilactobacillus sakei
Lautropia mirabilis
Lawsonella clevelandensis
Lawsonia intracellularis
Leclercia adecarboxylata
Legionella adelaidensis
Legionella anisa
Legionella birminghamensis
Legionella brunensis
Legionella cherrii
Legionella cincinnatiensis
Legionella clemsonensis
Legionella drancourtii
Legionella dumoffii
Legionella erythra
Legionella fairfieldensis
Legionella fallonii
Legionella feeleii
Legionella geestiana
Legionella gormanii
Legionella hackeliae
Legionella israelensis
Legionella jamestowniensis
Legionella jordanis
Legionella lansingensis
Legionella londiniensis
Legionella longbeachae
Legionella maceachernii
Legionella massiliensis
Legionella nautarum
Legionella norrlandica
Legionella oakridgensis
Legionella parisiensis
Legionella pneumophila
Legionella quateirensis
Legionella quinlivanii
Legionella rubrilucens
Legionella sainthelensi
Legionella santicrucis
Legionella shakespearei
Legionella spiritensis
Legionella steelei
Legionella tucsonensis
Legionella tunisiensis
Legionella wadsworthii
Legionella waltersii
Legionella worsleiensis
Leifsonia aquatica
Leifsonia xyli
Leminorella grimontii
Leminorella richardii
Leptospira alexanderi
Leptospira alstonii
Leptospira biflexa
Leptospira borgpetersenii
Leptospira broomii
Leptospira fainei
Leptospira inadai
Leptospira interrogans
Leptospira kirschneri
Leptospira kmetyi
Leptospira licerasiae
Leptospira mayottensis
Leptospira meyeri
Leptospira noguchii
Leptospira santarosai
Leptospira terpstrae
Leptospira vanthielii
Leptospira weilii
Leptospira wolbachii
Leptospira yanagawae
Leptotrichia buccalis
Leptotrichia goodfellowii
Leptotrichia shahii
Leptotrichia trevisanii
Leptotrichia wadei
Leuconostoc carnosum
Leuconostoc citreum
Leuconostoc lactis
Leuconostoc mesenteroides
Leuconostoc pseudomesenteroides
Levilactobacillus brevis
Ligilactobacillus salivarius
Limosilactobacillus fermentum
Listeria grayi
Listeria innocua
Listeria ivanovii
Listeria monocytogenes
Listeria seeligeri
Listeria welshimeri
Luteococcus peritonei
Luteococcus sanguinis
Lysinibacillus sphaericus
Mannheimia haemolytica
Massilia timonae
Megasphaera elsdenii
Megasphaera micronuciformis
Methylobacterium mesophilicum
Microbacterium
Microbacterium arborescens
Microbacterium foliorum
Microbacterium maritypicum
Microbacterium oxydans
Microbacterium paraoxydans
Microbacterium resistens
Microbacterium testaceum
Micrococcus luteus
Micrococcus luteus ATCC 49442
Micrococcus lylae
Mitsuokella multacida
Mobiluncus curtisii
Mobiluncus curtisii curtisii
Mobiluncus curtisii holmesii
Mobiluncus mulieris
Moellerella wisconsensis
Mogibacterium diversum
Mogibacterium neglectum
Mogibacterium timidum
Moraxella atlantae
Moraxella catarrhalis
Moraxella lacunata
Moraxella lincolnii
Moraxella nonliquefaciens
Moraxella osloensis
Morganella morganii
Morganella morganii morganii
Morganella morganii sibonii
Morococcus cerebrosus
Moryella indoligenes
Mycobacterium abscessus
Mycobacterium africanum
Mycobacterium alvei
Mycobacterium arupense
Mycobacterium asiaticum
Mycobacterium aurum
Mycobacterium avium
Mycobacterium barrassiae
Mycobacterium bohemicum
Mycobacterium bolletii
Mycobacterium bovis
Mycobacterium branderi
Mycobacterium brisbanense
Mycobacterium canariasense
Mycobacterium celatum
Mycobacterium chelonae
Mycobacterium chimaera
Mycobacterium chubuense
Mycobacterium colombiense
Mycobacterium conceptionense
Mycobacterium conspicuum
Mycobacterium cosmeticum
Mycobacterium diernhoferi
Mycobacterium doricum
Mycobacterium elephantis
Mycobacterium flavescens
Mycobacterium florentinum
Mycobacterium fortuitum
Mycobacterium franklinii
Mycobacterium gastri
Mycobacterium genavense
Mycobacterium goodii
Mycobacterium gordonae
Mycobacterium grossiae
Mycobacterium haemophilus
Mycobacterium hassiacum
Mycobacterium heckeshornense
Mycobacterium heidelbergense
Mycobacterium heraklionense
Mycobacterium hodleri
Mycobacterium holsaticum
Mycobacterium houstonense
Mycobacterium immunogenum
Mycobacterium interjectum
Mycobacterium intermedium
Mycobacterium intracellulare
Mycobacterium iranicum
Mycobacterium kansasii
Mycobacterium koreense
Mycobacterium kumamotonense
Mycobacterium kyorinense
Mycobacterium lentiflavum
Mycobacterium leprae
Mycobacterium lepromatosis
Mycobacterium llatzerense
Mycobacterium mageritense
Mycobacterium malmoense
Mycobacterium marinum
Mycobacterium massiliense
Mycobacterium microti
Mycobacterium monacense
Mycobacterium mucogenicum
Mycobacterium nebraskense
Mycobacterium neoaurum
Mycobacterium nonchromogenicum
Mycobacterium novocastrense
Mycobacterium obuense
Mycobacterium palustre
Mycobacterium paraffinicum
Mycobacterium parascrofulaceum
Mycobacterium peregrinum
Mycobacterium phlei
Mycobacterium phocaicum
Mycobacterium porcinum
Mycobacterium saopaulense
Mycobacterium scrofulaceum
Mycobacterium septicum
Mycobacterium setense
Mycobacterium sherrisii
Mycobacterium shigaense
Mycobacterium shimoidei
Mycobacterium simiae
Mycobacterium smegmatis
Mycobacterium szulgai
Mycobacterium talmoniae
Mycobacterium terrae
Mycobacterium thermoresistibile
Mycobacterium triplex
Mycobacterium triviale
Mycobacterium tuberculosis
Mycobacterium tusciae
Mycobacterium ulcerans
Mycobacterium wolinskyi
Mycobacterium xenopi
Mycolicibacterium aurum
Mycolicibacterium chlorophenolicum
Mycolicibacterium hassiacum
Mycolicibacterium vaccae
Mycolicibacterium wolinskyi
Mycoplasma amphoriforme
Mycoplasma capricolum
Mycoplasma faucium
Mycoplasma fermentans
Mycoplasma genitalium
Mycoplasma hominis
Mycoplasma hyopneumoniae
Mycoplasma orale
Mycoplasma penetrans
Mycoplasma pirum
Mycoplasma pneumoniae
Mycoplasma primatum
Mycoplasma salivarium
Mycoplasma spermatophilum
Mycoplasmopsis arginini
Mycoplasmopsis cynos
Mycoplasmopsis fermentans
Mycoplasmopsis pulmonis
Myroides marinus
Myroides odoratimimus
Myroides odoratus
Neisseria animaloris
Neisseria bacilliformis
Neisseria canis
Neisseria cinerea
Neisseria elongata
Neisseria elongata nitroreductens
Neisseria flavescens
Neisseria gonorrhoeae
Neisseria lactamica
Neisseria meningitidis
Neisseria mucosa
Neisseria polysaccharea
Neisseria sicca
Neisseria subflava
Neisseria wadsworthii
Neisseria weaveri
Neisseria zoodegmatis
Neorickettsia helminthoeca
Neorickettsia sennetsu
Nocardia abscessus
Nocardia acidivorans
Nocardia africana
Nocardia alba
Nocardia amamiensis
Nocardia anaemiae
Nocardia aobensis
Nocardia araoensis
Nocardia arizonensis
Nocardia arthritidis
Nocardia asiatica
Nocardia asteroides
Nocardia beijingensis
Nocardia brasiliensis
Nocardia brevicatena
Nocardia caishijiensis
Nocardia carnea
Nocardia cerradoensis
Nocardia concava
Nocardia coubleae
Nocardia crassostreae
Nocardia cummidelens
Nocardia cyriacigeorgica
Nocardia elegans
Nocardia exalbida
Nocardia farcinica
Nocardia flavorosea
Nocardia fusca
Nocardia gamkensis
Nocardia grenadensis
Nocardia harenae
Nocardia higoensis
Nocardia ignorata
Nocardia inohanensis
Nocardia jejuensis
Nocardia jiangxiensis
Nocardia kruczakiae
Nocardia lijiangensis
Nocardia mexicana
Nocardia mikamii
Nocardia miyunensis
Nocardia niigatensis
Nocardia ninae
Nocardia niwae
Nocardia nova
Nocardia otitidiscaviarum
Nocardia paucivorans
Nocardia pneumoniae
Nocardia pseudobrasiliensis
Nocardia pseudovaccinii
Nocardia puris
Nocardia rhamnosiphila
Nocardia salmonicida
Nocardia seriolae
Nocardia shimofusensis
Nocardia sienata
Nocardia soli
Nocardia speluncae
Nocardia takedensis
Nocardia tenerifensis
Nocardia terpenica
Nocardia testacea
Nocardia thailandica
Nocardia transvalensis
Nocardia uniformis
Nocardia vaccinii
Nocardia vermiculata
Nocardia veterana
Nocardia vinacea
Nocardia vulneris
Nocardia xishanensis
Nocardia yamanashiensis
Nocardiopsis dassonvillei
Ochrobactrum anthropi
Ochrobactrum intermedium
Ochrobactrum oryzae
Odoribacter laneus
Odoribacter splanchnicus
Oerskovia turbata
Oligella ureolytica
Oligella urethralis
Olsenella uli
Oribacterium sinus
Orientia tsutsugamushi
Oscillibacter ruminantium
Paenalcaligenes hominis
Paenibacillus alvei
Paenibacillus macerans
Paenibacillus mucilaginosus
Paenibacillus polymyxa
Paenibacillus popilliae
Paeniclostridium sordellii
Pandoraea apista
Pandoraea pulmonicola
Pandoraea sputorum
Pannonibacter phragmitetus
Pantoea agglomerans
Pantoea ananatis
Pantoea dispersa
Parabacteroides distasonis
Parabacteroides faecis
Parabacteroides goldsteinii
Parabacteroides gordonii
Parabacteroides johnsonii
Parabacteroides massiliensis
Parabacteroides merdae
Paraburkholderia fungorum
Parachlamydia acanthamoebae
Paraclostridium bifermentans
Paracoccus sanguinis
Paracoccus yeei
Paraeggerthella hongkongensis
Parascardovia denticolens
Parvimonas micra
Pasteurella aerogenes
Pasteurella bettyae
Pasteurella canis
Pasteurella dagmatis
Pasteurella gallinarum
Pasteurella haemolytica
Pasteurella multocida
Pasteurella multocida multocida
Pasteurella multocida septica
Pediococcus acidilactici
Pediococcus pentosaceus
Pelobacter propionicus
Peptococcus niger
Peptoniphilus asaccharolyticus
Peptoniphilus coxii
Peptoniphilus duerdenii
Peptoniphilus harei
Peptoniphilus indolicus
Peptoniphilus lacrimalis
Peptostreptococcus anaerobius
Peptostreptococcus canis
Peptostreptococcus stomatis
Photobacterium damselae
Photorhabdus asymbiotica
Photorhabdus luminescens
Plesiomonas shigelloides
Pluralibacter gergoviae
Porphyromonas asaccharolytica
Porphyromonas catoniae
Porphyromonas endodontalis
Porphyromonas gingivalis
Porphyromonas gingivicanis
Porphyromonas somerae
Porphyromonas uenonis
Prevotella bergensis
Prevotella bivia
Prevotella buccae
Prevotella buccalis
Prevotella corporis
Prevotella dentalis
Prevotella denticola
Prevotella disiens
Prevotella intermedia
Prevotella loescheii
Prevotella melaninogenica
Prevotella multiformis
Prevotella multisaccharivorax
Prevotella nigrescens
Prevotella oralis
Prevotella oris
Prevotella tannerae
Prevotella timonensis
Propionibacterium acidifaciens
Propionibacterium propionicum
Propionimicrobium lymphophilum
Proteus mirabilis
Proteus penneri
Proteus vulgaris
Providencia alcalifaciens
Providencia rettgeri
Providencia rustigianii
Providencia stuartii
Pseudomonas aeruginosa
Pseudomonas alcaligenes
Pseudomonas cannabina
Pseudomonas citronellolis
Pseudomonas fluorescens
Pseudomonas fulva
Pseudomonas luteola
Pseudomonas mendocina
Pseudomonas monteilii
Pseudomonas mosselii
Pseudomonas oryzihabitans
Pseudomonas otitidis
Pseudomonas poae
Pseudomonas protegens
Pseudomonas pseudoalcaligenes
Pseudomonas putida
Pseudomonas stutzeri
Pseudomonas veronii
Pseudopropionibacterium propionicum
Pseudoramibacter
Pseudoramibacter alactolyticus
Psychrobacter cryohalolentis
Psychrobacter immobilis
Psychrobacter phenylpyruvicus
Rahnella aquatilis
Ralstonia insidiosa
Ralstonia mannitolilytica
Ralstonia pickettii
Ralstonia solanacearum
Raoultella ornithinolytica
Raoultella planticola
Raoultella terrigena
Rhodococcus equi
Rhodococcus erythropolis
Rhodococcus fascians
Rhodococcus rhodochrous
Rickettsia africae
Rickettsia akari
Rickettsia amblyommatis
Rickettsia australis
Rickettsia canadensis
Rickettsia conorii
Rickettsia felis
Rickettsia japonica
Rickettsia massiliae
Rickettsia monacensis
Rickettsia parkeri
Rickettsia prowazekii
Rickettsia raoultii
Rickettsia rickettsii
Rickettsia sibirica
Rickettsia slovaca
Rickettsia typhi
Riemerella anatipestifer
Robinsoniella peoriensis
Roseobacter denitrificans
Roseomonas cervicalis
Roseomonas gilardii
Roseomonas mucosa
Rothia aeria
Rothia dentocariosa
Rothia mucilaginosa
Rouxiella chamberiensis
Ruminococcus flavefaciens
Salmonella bongori
Salmonella enterica
Salmonella enterica ssp. Arizonae
Salmonella enterica ssp. Diarizonae
Salmonella enterica ssp. Enterica
Salmonella enteritidis
Salmonella paratyphi
Salmonella typhi
Salmonella typhimurium
Sanguibacteroides justesenii
Scardovia inopinata
Scardovia wiggsiae
Selenomonas artemidis
Selenomonas flueggei
Selenomonas infelix
Selenomonas noxia
Selenomonas sputigena
Serratia ficaria
Serratia fonticola
Serratia grimesii
Serratia liquefaciens
Serratia marcescens
Serratia odorifera
Serratia plymuthica
Serratia proteamaculans
Serratia quinivorans
Serratia rubidaea
Serratia ureilytica
Shewanella algae
Shewanella putrefaciens
Shigella boydii
Shigella dysenteriae
Shigella flexneri
Shigella sonnei
Shimwellia blattae
Siccibacter turicensis
Simkania negevensis
Slackia exigua
Sneathia sanguinegens
Sphingobacterium multivorum
Sphingobacterium spiritivorum
Sphingobium yanoikuyae
Sphingomonas paucimobilis
Staphylococcus agnetis
Staphylococcus argenteus
Staphylococcus arlettae
Staphylococcus aureus
Staphylococcus auricularis
Staphylococcus capitis
Staphylococcus capitis capitis
Staphylococcus capitis ureolyticus
Staphylococcus caprae
Staphylococcus carnosus
Staphylococcus chromogenes
Staphylococcus cohnii
Staphylococcus cohnii cohnii
Staphylococcus cohnii urealyticus
Staphylococcus condimenti
Staphylococcus delphini
Staphylococcus epidermidis
Staphylococcus equorum
Staphylococcus gallinarum
Staphylococcus haemolyticus
Staphylococcus hominis
Staphylococcus hominis hominis
Staphylococcus hominis novobiosepticius
Staphylococcus hyicus
Staphylococcus intermedius
Staphylococcus lugdunensis
Staphylococcus massiliensis
Staphylococcus pasteuri
Staphylococcus pettenkoferi
Staphylococcus pseudintermedius
Staphylococcus saccharolyticus
Staphylococcus saprophyticus
Staphylococcus schleiferi
Staphylococcus schleiferi coagulans
Staphylococcus schleiferi schleiferi
Staphylococcus sciuri
Staphylococcus simiae
Staphylococcus simulans
Staphylococcus succinus
Staphylococcus vitulinus
Staphylococcus warneri
Staphylococcus xylosus
Stenotrophomonas acidaminiphila
Stenotrophomonas maltophilia
Streptobacillus moniliformis
Streptococcus acidominimus
Streptococcus agalactiae
Streptococcus anginosus
Streptococcus canis
Streptococcus constellatus
Streptococcus constellatus constellatus
Streptococcus constellatus pharyngis
Streptococcus criceti
Streptococcus cristatus
Streptococcus dentisani
Streptococcus dysgalactiae
Streptococcus dysgalactiae dysgalactiae
Streptococcus dysgalactiae equisimilis
Streptococcus equi
Streptococcus equi equi
Streptococcus equi zooepidemicus
Streptococcus equinus
Streptococcus ferus
Streptococcus gallolyticus
Streptococcus gallolyticus ssp. Gallolyticus
Streptococcus gallolyticus ssp. Pateurianus
Streptococcus gordonii
Streptococcus hyovaginalis
Streptococcus infantarius
Streptococcus infantis
Streptococcus iniae
Streptococcus intermedius
Streptococcus lutetiensis
Streptococcus macacae
Streptococcus macedonicus
Streptococcus massiliensis
Streptococcus mitis
Streptococcus mutans
Streptococcus oralis
Streptococcus parasanguinis
Streptococcus pasteurianus
Streptococcus peroris
Streptococcus pneumoniae
Streptococcus porcinus
Streptococcus pseudopneumoniae
Streptococcus pseudoporcinus
Streptococcus pyogenes
Streptococcus ratti
Streptococcus salivarius
Streptococcus sanguinis
Streptococcus sinensis
Streptococcus sobrinus
Streptococcus suis
Streptococcus thermophilus
Streptococcus tigurinus
Streptococcus uberis
Streptococcus urinalis
Streptococcus vestibularis
Streptomyces bikiniensis
Streptomyces cattleya
Streptomyces griseus
Streptomyces somaliensis
Succinivibrio dextrinosolvens
Sutterella wadsworthensis
Suttonella indologenes
Tannerella forsythia
Tatumella ptyseos
Taylorella asinigenitalis
Taylorella equigenitalis
Tissierella praeacuta
Treponema amylovorum
Treponema denticola
Treponema lecithinolyticum
Treponema maltophilum
Treponema medium
Treponema pallidum
Treponema parvum
Treponema pectinovorum
Treponema pertenue
Treponema putidum
Treponema socranskii
Treponema vincentii
Tropheryma whipplei
Trueperella pyogenes
Tsukamurella paurometabola
Tsukamurella pulmonis
Tsukamurella tyrosinosolvens
Turicella otitidis
Ureaplasma parvum
Ureaplasma urealyticum
Vagococcus fluvialis
Veillonella dispar
Veillonella montpellierensis
Veillonella parvula
Veillonella seminalis
Vibrio alginolyticus
Vibrio cholerae
Vibrio cincinnatiensis
Vibrio fluvialis
Vibrio furnissii
Vibrio harveyi
Vibrio metschnikovii
Vibrio mimicus
Vibrio navarrensis
Vibrio parahaemolyticus
Vibrio vulnificus
Waddlia chondrophila
Wautersiella falsenii
Weeksella virosa
Weissella confusa
Weissella paramesenteroides
Weissella viridescens
Williamsia muralis
Wohlfahrtiimonas chitiniclastica
Wolbachia pipientis
Xanthomonas axonopodis
Xanthomonas campestris
Xylanimonas cellulosilytica
Yersinia bercovieri
Yersinia enterocolitica
Yersinia frederiksenii
Yersinia intermedia
Yersinia kristensenii
Yersinia pestis
Yersinia pseudotuberculosis
Yersinia ruckeri
Yokenella regensburgei

The present disclosure also relates to methods and systems that use computer-generated information to design and/or construct a database of probe sequences or set of probes. For example, in some embodiments, a first analytical tool using the information from species-specific or clade-specific marker gene sequences and/or 16S ribosomal RNA sequences and/or virulence factor sequences and/or AMR genes and a second analytical tool to fragment the sequences into oligonucleotides with the desired or advantageous parameters for the probes, including but not limited to length, distance spaced between the probes on the target sequences, and percentage sequence identity.

In a further aspect, analytical tools such as a first module configured to perform the choice of species-specific or clade-specific marker gene sequences and/or 16S ribosomal RNA sequences and/or virulence factor sequences and/or AMR genes, and a second module to perform the fragmentation of the sequences may be provided that determines desired or advantageous features of the oligonucleotides such as the length, distance spaced between the oligonucleotides on the sequences, and/or percentage sequence identity. The results of these tools form a model for use in designing the oligonucleotides for the disclosed database of probe sequences or set of probes.

An illustrative system for generating a design model includes an analytical tool such as a module configured to include species-specific or clade-specific marker gene sequences extracted from the Metaphlan4 database 16S ribosomal RNA sequences extracted from SILVA database for a total of 1333 bacterial species, virulence factor sequences extracted from the VFDB, and/or AMR extracted from CARD. The analytical tool may include any suitable hardware, software, or combination thereof for determining correlations. A second analytical tool such as module is used to fragment the sequences. This analytical tool may include any suitable hardware, software, or combination for determining the desired or advantageous features of the oligonucleotides including but not limited to length, distance spaced between the probes on the sequences, and percentage sequence identity.

After the sequence information is obtained for the oligonucleotide probes, the oligonucleotides can be synthesized by any method known in the art including but not limited to solid-phase synthesis using phosphoramidite method and phosphoramidite building blocks derived from protected 2′-deoxynucleosides (dA, dC, dG, and T), ribonucleosides (A, C, G, and U), or chemically modified nucleosides, e.g. linked nucleic acids (LNA), bridged nucleic acids (BNA) or peptide nucleic acids (PNA).

One embodiment is a library or platform comprising the set of oligonucleotide probes with the sequences in the database that is capable of capturing nucleic acids from at least one bacterium. In some embodiments, the library comprising the oligonucleotide probes is capable of capturing nucleic acids from more than one bacteria. In some embodiments, the library comprising the oligonucleotide probes is capable of capturing nucleic acids from more than ten bacteria. In some embodiments, the library comprising the oligonucleotide probes is capable of capturing nucleic acids from more than fifty bacteria. In some embodiments, the library comprising the oligonucleotide probes is capable of capturing nucleic acids from more than one hundred bacteria. In some embodiments, the library comprising the oligonucleotide probes is capable of capturing nucleic acids from more than one hundred and fifty bacteria. In some embodiments, the library comprising the oligonucleotide probes is capable of capturing nucleic acids from more than two hundred bacteria. In some embodiments, the library comprising the oligonucleotide probes is capable of capturing nucleic acids from more than two hundred and fifty bacteria. In some embodiments, the library or platform comprising the oligonucleotide probes is capable of capturing nucleic acids from more than three hundred bacteria. In some embodiments, the library comprising the oligonucleotide probes is capable of capturing nucleic acids from more than four hundred bacteria. In some embodiments, the library comprising the oligonucleotide probes is capable of capturing nucleic acids from more than five hundred bacteria. In some embodiments, the library comprising the oligonucleotide probes is capable of capturing nucleic acids from more than six hundred bacteria. In some embodiments, the library comprising the oligonucleotide probes is capable of capturing nucleic acids from more than seven hundred bacteria. In some embodiments, the library comprising the oligonucleotide probes is capable of capturing nucleic acids from more than eight hundred bacteria. In some embodiments, the library comprising the oligonucleotide probes is capable of capturing nucleic acids from more than nine hundred bacteria. In some embodiments, the library comprising the oligonucleotide probes is capable of capturing nucleic acids from more than one thousand hundred bacteria.

In one embodiment, the oligonucleotides are in solution.

In one embodiment, the oligonucleotides are pre-bound to a solid support or substrate. Preferred solid supports include, but are not limited to, beads (e.g., magnetic beads (i.e., the bead itself is magnetic, or the bead is susceptible to capture by a magnet)) made of metal, glass, plastic, dextran (such as the dextran bead sold under the tradename, Sephadex (Pharmacia)), silica gel, agarose gel (such as those sold under the tradename, Sepharose (Pharmacia)), or cellulose); capillaries; flat supports (e.g., filters, plates, or membranes made of glass, metal (such as steel, gold, silver, aluminum, copper, or silicon), or plastic (such as polyethylene, polypropylene, polyamide, or polyvinylidene fluoride)); a chromatographic substrate; a microfluidics substrate; and pins (e.g., arrays of pins suitable for combinatorial synthesis or analysis of beads in pits of flat surfaces (such as wafers), with or without filter plates). Additional examples of suitable solid supports include, without limitation, agarose, cellulose, dextran, polyacrylamide, polystyrene, sepharose, and other insoluble organic polymers. Appropriate binding conditions (e.g., temperature, pH, and salt concentration) may be readily determined by the skilled artisan.

The oligonucleotides may be either covalently or non-covalently bound to the solid support. Furthermore, the oligonucleotides may be directly bound to the solid support (e.g., the oligonucleotides are in direct van der Waal and/or hydrogen bond and/or salt-bridge contact with the solid support), or indirectly bound to the solid support (e.g., the oligonucleotides are not in direct contact with the solid support themselves). Where the oligonucleotides are indirectly bound to the solid support, the nucleotides of the capture nucleic acid are linked to an intermediate composition that, itself, is in direct contact with the solid support.

To facilitate binding of the oligonucleotides to the solid support, the oligonucleotides may be modified with one or more molecules suitable for direct binding to a solid support and/or indirect binding to a solid support by way of an intermediate composition or spacer molecule that is bound to the solid support (such as an antibody, a receptor, a binding protein, or an enzyme). Examples of such modifications include, without limitation, a ligand (e.g., a small organic or inorganic molecule, a ligand to a receptor, a ligand to a binding protein or the binding domain thereof (such as biotin and digoxigenin)), an antigen and the binding domain thereof, an aptamer, a peptide tag, an antibody, and a substrate of an enzyme. In a preferred embodiment, the oligonucleotides comprise biotin.

Linkers or spacer molecules suitable for spacing biological and other molecules, including nucleic acids/polynucleotides, from solid surfaces are well-known in the art, and include, without limitation, polypeptides, saturated or unsaturated bifunctional hydrocarbons, and polymers (e.g., polyethylene glycol). Other useful linkers are commercially available.

In a further embodiment, the sequences of the oligonucleotides are the complement of (i.e., is complementary to) a sequence of the marker sequences of one or more bacteria as well as AMR genes and/or virulence factors and/or 16S ribosomal RNA. In another embodiment, the oligonucleotides are capable of hybridizing to a sequence of the marker sequences of one or more bacteria as well as AMR genes and/or virulence factors and/or 16S ribosomal RNA under stringent conditions.

The “complement” of a nucleic acid sequence refers, herein, to a nucleic acid molecule which is completely complementary to another nucleic acid, or which will hybridize to the other nucleic acid under conditions of high stringency. High-stringency conditions are known in the art. Sec, e.g., Maniatis et al., Molecular Cloning: A Laboratory Manual, 2nd ed. (Cold Spring Harbor: Cold Spring Harbor Laboratory, 1989) and Ausubel et al., eds., Current Protocols in Molecular Biology (New York, N.Y.: John Wiley & Sons, Inc., 2001). Stringent conditions are sequence-dependent, and may vary depending upon the circumstances.

In one embodiment, the oligonucleotides are synthesized using a cleavable programmable array. The oligonucleotides are cleaved from the array and hybridized with the nucleic acids from the sample in solution.

The set of probes can be in the form of a collection of oligonucleotides, preferably designed as set forth above, i.e., a probe library. The oligonucleotides can be in solution or attached to a solid state, such as an array or a bead. Additionally, the oligonucleotides can be modified with another molecule. In a preferred embodiment, the oligonucleotides comprise biotin.

The database of probe sequences can also be in the form of a database or databases which can include information regarding the sequence and length of each oligonucleotide probe, and the bacterium and/or marker sequence from which the oligonucleotide sequence derived as well as AMR genes and virulence factors and 16S ribosomal RNA. The database can searchable. From the database, one of skill in the art can obtain the information needed to design and synthesis the oligonucleotide probes. The databases can also be recorded on machine-readable storage medium, any medium that can be read and accessed directly by a computer. A machine-readable storage medium can comprise, for example, a data storage material that is encoded with machine-readable data or data arrays. Machine-readable storage medium can include but are not limited to magnetic storage media, optical storage media, electrical storage media, and hybrids. One of skill in the art can easily determine how presently known machine-readable storage medium and future developed machine-readable storage medium can be used to create a manufacture of a recording of any database information. “Recorded” refers to a process for storing information on a machine-readable storage medium using any method known in the art.

### Construction of a Sequencing Library

A further embodiment of the present disclosure is a method of constructing a sequencing library suitable for sequencing with any high throughput sequencing method utilizing the set of probes.

Accordingly, the method may include the following steps.

Nucleic acids from a sample are obtained. The sample used in the present methods may be an environmental sample, a food sample, or a biological sample. The preferred sample is a biological sample or an environmental sample (e.g., a wastewater sample or sewage sample). A biological sample may be obtained from a tissue of a subject or bodily fluid from a subject including, but not limited to, nasopharyngeal aspirate, blood, cerebrospinal fluid, saliva, serum, urine, sputum, bronchial lavage, pericardial fluid, or peritoneal fluid, or a solid such as feces. A biological sample can also be cells, cell culture or cell culture medium. The sample may or may not comprise or contain any bacterial nucleic acids. In one embodiment, the sample is from a vertebrate subject, and in a further embodiment, the sample is from a human subject. In another embodiment, the sample comprises blood. In another preferred embodiment, the sample comprises cells, cell culture, cell culture medium or any other composition being used for developing pharmaceutical and therapeutic agents. In some embodiments, the sample is from food or a food supply.

The nucleic acids from the sample are subjected to fragmentation, to obtain a nucleic acid fragment. There are no special limitations on the type of the nucleic acid sample which may be used and there are no special limitations on means for performing the fragmentation. Any chemical or physical method which randomly fragments nucleic acid samples may be used. It is preferred that the nucleic acid sample is fragmented to obtain a nucleic acid fragment having a length of about 200 bp to about 300 bp or any other size distribution suitable for the respective sequencing platform.

After being obtained, the nucleic acid fragments can be ligated to an adaptor. In one embodiment, the adaptor is a linear adaptor. Linear adaptors can be added to the fragments by end-repairing the fragments, to obtain an end-repaired fragment; adding an adenine base to the 3′ ends of the fragment, to obtain a fragment having an adenine at the 3′ end; and ligating an adaptor to the fragment having an adenine at the 3′end.

In some embodiments, the adaptor comprises an identifier sequence. In some embodiments, the adaptor comprises sequences for priming for amplification. In some embodiments, the adaptor comprises both an identified sequence and sequences for priming for amplification.

After the nucleic acid fragment is ligated to the adaptor, it is contacted with the oligonucleotide probes described herein, under conditions that allow the nucleic acid fragment to hybridize to the oligonucleotide probes if the nucleic acid comprises any sequences from bacteria or genes represented in the database, set of sequences, or oligonucleotide probes described herein. This step may be performed in solution or in a solid phase hybridization method.

After contact with the oligonucleotides, any hybridization product(s) may be subject to amplification conditions. In one embodiment, primers for amplification are present in the adaptor ligated to the nucleic acid fragment. The resulting amplified product(s) comprise the sequencing library that is suitable to be sequenced using any HTS system now known or later developed.

Amplification may be carried out by any means known in the art, including polymerase chain reaction (PCR) and isothermal amplification. PCR is a practical system for in vitro amplification of a DNA base sequence. For example, a PCR assay may use a heat-stable polymerase and two primers: one complementary to the (+)-strand at one end of the sequence to be amplified; and the other complementary to the (−)-strand at the other end. Because the newly-synthesized DNA strands can subsequently serve as additional templates for the same primer sequences, successive rounds of primer annealing, strand elongation, and dissociation may produce rapid and highly-specific amplification of the desired sequence. PCR also may be used to detect the existence of a defined sequence in a DNA sample. In one embodiment, the hybridization products are mixed with suitable PCR reagents. A PCR reaction is then performed to amplify the hybridization products.

In one embodiment, the sequencing library is constructed using the probe set in a cleavable array. Nucleic acids from the sample are extracted and subjected to reverse transcriptase treatment and ligated to an adaptor comprising an identifier and sequences for priming for amplification. The oligonucleotides are synthesized using a cleavable array platform wherein the oligonucleotides are biotinylated. The biotinylated oligonucleotides are then cleaved from the solid matrix into solution with the nucleic acids from the sample to enable hybridization of the oligonucleotides to any bacterial nucleic acids in solution. After hybridization, nucleic acid(s) from the sample bound to the biotinylated oligonucleotides comprising the probe set, i.e., hybridization product(s), is collected by streptavidin magnetic beads, and amplified by PCR using the adaptor sequences as specific priming sites, resulting in an amplified product for sequencing on any known HTS systems (Ion, Illumina, 454) and any HTS system developed in the future.

In some embodiments, a sample comprising nucleic acids is exposed to the oligonucleotide probes described under hybridization conditions. After hybridization, the probes are captured (e.g., biotinylated probes are captured on streptavidin magnetic beads) and hybridization products are purified. Nucleic acids which bound the probes can be released and subsequently prepared for amplification and/or HTS sequencing, for example, by adding adaptor sequence portions to the released nucleic acids and/or size selecting the released nucleic acids.

In a further embodiment, the sequencing library can be directly sequenced using any method known in the art. In other words, the nucleic acids captured by the probes can be sequenced without amplification.

### Methods and Systems Using the Disclosed Database of Sequences and Set of Probes

The present disclosure includes methods and systems for the detection, identification and/or differentiation of bacteria and/or pathogenicity elements, and/or AMR genes, and/or 16S ribosomal RNA, in any sample, utilizing the database of probe sequences or set of probes.

The methods and systems may be used to detect bacteria and/or pathogenicity elements and/or AMR and/or 16S ribosomal RNA genes, in research, clinical, environmental, and food samples. Additional applications include, without limitation, detection of infectious pathogens, the screening of blood products (e.g., screening blood products for infectious agents), biodefense, food safety, environmental contamination, forensics, and genetic-comparability studies. The present disclosure also provides methods and systems for detecting bacteria and/or pathogenicity elements and/or AMR genes and/or 16S ribosomal RNA in cells, cell culture, cell culture medium and other compositions used for the development of pharmaceutical and therapeutic agents. Accordingly, the present disclosure provides methods and systems for a myriad of specific applications, including, without limitation, a method for determining the presence of bacteria and/or pathogenicity elements and/or AMR genes, and/or 16S ribosomal RNA, in a sample, a method for screening blood products, a method for assaying a food product for contamination, a method for assaying a sample for environmental contamination, and a method for detecting genetically-modified organisms. The present disclosure further provides use of the system in such general applications as biodefense against bioterrorism, forensics, and genetic-comparability studies.

The subject may be any animal, particularly a vertebrate and more particularly a mammal or avian, including, without limitation, a cow, dog, human, monkey, mouse, pig, rat, chicken or wildlife species such as a bat or a rodent. The subject may also be an invertebrate such as tick, mosquito or sand fly. In some embodiments, the subject is a human. The subject may be known to have a pathogen infection, suspected of having a pathogen infection, or believed not to have a pathogen infection.

The systems and methods described herein support the multiplex detection of multiple bacteria and bacterial transcripts in any sample.

Thus, one embodiment provides a system for the detection, identification and/or differentiation of bacteria and/or pathogenicity elements and/or AMR genes and/or 16S ribosomal RNA, in any sample. The system includes at least one subsystem wherein the subsystem includes the database of probe sequences or set of oligonucleotide probes as described herein. The system can also include additional subsystems for the purpose of: preparation of oligonucleotides from the database of probe sequences; isolation and preparation of the nucleic acid from the sample; hybridization of the nucleic acid from the sample with the oligonucleotides to form hybridization product(s); amplification of the hybridization product(s); sequencing the hybridization product(s); amplification of the nucleic acid(s) from the sample which do not form hybridization product(s); sequencing the nucleic acid(s) from the sample which do not form hybridization product(s); and identification and characterization of the bacteria, and/or pathogenicity elements and/or AMR genes and/or 16S ribosomal RNA by the comparison between the sequences of the hybridization product(s) and/or nucleic acids, and known bacteria and/or pathogenicity elements and/or AMR genes and/or 16S ribosomal RNA.

Additionally, the present disclosure provides a method for the detection, identification, and/or differentiation of bacteria and/or pathogenicity elements and/or AMR genes, and/or 16S ribosomal RNA, in any sample, including the steps of: obtaining the sample; isolating and preparing the nucleic acid from the sample; contacting the nucleic acid or derivatives thereof from the sample with the oligonucleotides generated from the disclosed database of probe sequences or set of oligonucleotide probes as described herein under conditions sufficient for the nucleic acid fragments and the oligonucleotides to hybridize; and detecting any hybridization products formed between the nucleic acid and the oligonucleotides.

These methods can also include additional steps to: amplify hybridization product(s); sequence the hybridization product(s); amplify nucleic acid(s) from the sample which do not form hybridization product(s); sequence nucleic acid(s) or derivatives thereof from the sample which do not form hybridization product(s); and comparison of hybridization product(s) and/or nucleic acid(s) from the sample which do not form hybridization product(s) with sequences of known bacteria, 16S ribosomal RNA, AMR genes and/or pathogenicity elements.

As disclosed above, the methods can be performed on any sample, including but not limited to biological samples, environmental samples, or food samples. One such sample is a biological sample. A biological sample may be obtained from a tissue of a subject or bodily fluid from a subject including but not limited to nasopharyngeal aspirate, blood, cerebrospinal fluid, saliva, serum, urine, sputum, bronchial lavage, pericardial fluid, or peritoneal fluid, or a solid such as feces. A biological sample can also be cells, cell culture or cell culture medium. The sample may or may not comprise or contain any bacterial nucleic acids.

In one embodiment, the sample is from a vertebrate subject, and in a further embodiment, the sample is from a human subject. In another embodiment, the sample is from an invertebrate subject.

In another embodiment, the sample comprises cells, cell culture, cell culture medium or any other composition being used for developing pharmaceutical and therapeutic agents.

In some embodiments, the nucleic acids from the sample are further processed by shearing, adaptor, etc., forming derivatives of the isolated nucleic acid.

### Kits

The disclosure also includes reagents and kits for practicing the disclosed methods. These reagents and kits may vary.

One reagent would be the disclosed set of probes, which can be in the form of a collection of oligonucleotide probes which comprise sequences derived from the disclosed database of probe sequences. This collection of oligonucleotide probes can be in solution or attached to a solid state. Additionally, the oligonucleotide probes can be modified for use in a reaction. A preferred modification is the addition of biotin to the probes.

A further reagent is a searchable database with information regarding the oligonucleotides including at least sequence information, length, and the origin.

Other reagents in the kit could include reagents for isolating and preparing nucleic acids from a sample, hybridizing the nucleic acid fragments from the sample with the oligonucleotides of the probe set, amplifying the hybridization products, and obtaining sequence information.

Kits may include any of the above-mentioned reagents, as well as reference/control sequences that can be used to compare the test sequence information obtained, by for example, suitable computing means based upon an input of sequence information.

In addition, kits would also further include instructions.

A further embodiment is a kit for designing and/or constructing the database of probe sequences comprising analytical tools to choose sequence information and break the sequences into fragments for oligonucleotides with the proper parameters including proper length, distance spaced between the oligonucleotides on the target sequences, and percentage sequence identity. This kit could also include instructions as to database and target sequence choice.

### Additional Embodiments

According to embodiments of the present invention, there is provided a bacterial sequence capture platform for the detection, identification, and/or differentiation of bacterially-derived sequences in a sample,
the platform comprising a plurality of oligonucleotide probes, wherein the plurality comprises at least one oligonucleotide probe which comprises a hybridization portion partially or fully complementary to a portion of a bacterially-derived sequence selected from the group consisting of a bacterial gene sequence, a 16S ribosomal RNA sequence, a pathogenicity element sequence, a virulence factor sequence, and an antimicrobial resistance (AMR) gene sequence,
wherein the sequences of the hybridization portions of the oligonucleotide probes cluster at about 90-100% sequence identity,
wherein each hybridization portion of an oligonucleotide probe is about 5-300 nucleotides in length,
wherein different hybridization portions that each bind a different portion of the same bacterially-derived sequence are tiled across said bacterially-derived sequence and have an inter-probe spacing of about 20-100 nucleotides, and
wherein the plurality of oligonucleotide probes of the platform comprises 100,000 to 1,000,000 oligonucleotide probes, preferably less than about 1,000,000 oligonucleotide probes.

In some embodiments, each hybridization portion of an oligonucleotide probe is about 50-200 nucleotides in length, preferably about 100-150 nucleotides in length, more preferably about 120 nucleotides in length.

In some embodiments, the average length of the plurality of hybridization portions of oligonucleotide probes is about 120 nucleotides.

In some embodiments, different hybridization portions that each bind a different portion of the same bacterially-derived sequence are tiled across said bacterially-derived sequence and have an inter-probe spacing of about 60 nucleotides.

In some embodiments, the sequences of the hybridization portions of the oligonucleotide probes cluster at about 90%, 91%, 92%, 93%, 94%, 95%, 96%, 97%, 98%, 99%, or 100% sequence identity.

In some embodiments, the plurality of oligonucleotide probes comprises hybridization portions partially or fully complementary to portions of bacterially-derived sequences comprising one or more bacterial gene sequences, one or more 16S ribosomal RNA sequences, one or more pathogenicity element sequences, one or more virulence factor sequences, and/or one or more antimicrobial resistance (AMR) gene sequences.

In some embodiments, the bacterial gene sequence is a species-specific or clade-specific gene sequence.

In some embodiments, the species-specific or clade-specific gene sequences are obtained from Metaphlan4 database.

In some embodiments, the 16S ribosomal RNA sequences are obtained from the SILVA database.

In some embodiments, the virulence factor sequences are obtained from the Virulence Factor Database (VFDB).

In some embodiments, the AMR genes are obtained from the Comprehensive Antibiotic Resistance Database (CARD).

In some embodiments, each bacterially-derived sequence comprises a portion that is about 50-300 nucleotides in length and is partially or fully complementary to a hybridization portion of an oligonucleotide probe.

In some embodiments, each hybridization portion is at least 90%, 91%, 92%, 93%, 94%, 95%, 96%, 97%, 98%, 99% or 100% complementary to a portion of a bacterially-derived sequence.

In some embodiments, the plurality of oligonucleotide probes comprises at least one oligonucleotide probe which comprises a hybridization portion partially or fully complementary to a portion of a bacterially-derived sequence from a bacterial species listed in Table 1.

In some embodiments, every bacterial species listed in Table 1 comprises a sequence, preferably a unique sequence relative to any other bacterial species listed in Table 1, that is partially or fully complementary to a hybridization portion of a oligonucleotide probe of the plurality of the platform.

In some embodiments, each oligonucleotide probe comprises a capture portion.

In some embodiments, the capture portion is selected from the group consisting of biotin, digoxygenin, a ligand, a small organic molecule, a small inorganic molecule, an aptamer, an antigen, an antibody, and a substrate.

In some embodiments, each oligonucleotide probe is biotinylated.

In some embodiments, and means for capturing, isolating, and/or purifying the plurality of oligonucleotide probes from a mixture of other nucleic acid molecules.

In some embodiments, the oligonucleotide probes comprise DNA, RNA, bridged nucleic acids, locked nucleic acids, and/or peptide nucleic acids. In some embodiments, the hybridization portion of an oligonucleotide probe comprises DNA, RNA, bridged nucleic acids, locked nucleic acids, and/or peptide nucleic acids. In some embodiments, the oligonucleotide probes are capable of hybridizing DNA, cDNA, RNA, and/or mRNA molecules.

In some embodiments, the oligonucleotide probes of the platform may be in solution or attached to a solid support. In some embodiments, the platform comprises oligonucleotide probes generated in an array format, e.g., a cleavable array format. In some embodiments, the platform comprises oligonucleotide probes generated from semiconductor-based synthetic DNA manufacturing.

In some embodiments, the sample is a biological sample or an environmental sample.

In some embodiments, the sample is selected from the group consisting of saliva, mucus, a nasopharyngeal swab, serum, plasma, blood, urine, feces, cerebrospinal fluid, a bodily fluid, cultured cells, an organ tissue, and biopsied tissue.

In some embodiments, the sample is selected from the group consisting of an aqueous sample, a liquid sample, water, wastewater, sewage, greywater, blackwater, freshwater, liquid waste, seawater, drinking water, air, a gaseous sample, soil, a food sample, culture medium, and a swab of an inanimate surface or object.

In some embodiments, the sample is obtained from a sewage system, a drainage system, a plumbing system, or a water treatment facility.

In some embodiments, the sample is obtained from a human subject.

According to embodiments of the present invention, there is provided a method of screening a sample for bacterially-derived sequences, the method comprising:
a) exposing the sample, or nucleic acids isolated, amplified, and/or enriched from the sample, to any one of the bacterial sequence capture platforms described herein to form one or more hybridization products, wherein each hybridization product comprises a nucleic acid of the sample and an oligonucleotide probe of the platform;
b) capturing the one or more hybridization products; and
c) identifying the presence of one or more bacterially-derived sequences in the sample based on the sequences of the one or more captured hybridization products;
thereby screening the sample for bacterially-derived sequences.

In some embodiments, nucleic acids in the sample are isolated and/or enriched prior to the exposing in step (a).

In some embodiments, the sample is a biological sample or an environmental sample.

In some embodiments, the sample is selected from the group consisting of saliva, mucus, a nasopharyngeal swab, serum, plasma, blood, urine, feces, cerebrospinal fluid, a bodily fluid, cultured cells, an organ tissue, and biopsied tissue.

In some embodiments, the sample is selected from the group consisting of an aqueous sample, a liquid sample, water, wastewater, sewage, greywater, blackwater, freshwater, liquid waste, seawater, drinking water, air, a gaseous sample, soil, a food sample, culture medium, and a swab of an inanimate surface or object.

In some embodiments, the sample is obtained from a sewage system, a drainage system, a plumbing system, or a water treatment facility.

In some embodiments, the sample is obtained from a human subject.

In some embodiments, the method further comprises:
sequencing one or more detected hybridization products;
comparing the nucleotide sequence of the one or more hybridization products to nucleotide sequences of known bacterially-derived sequences; and
identifying and/or differentiating one or more bacterially-derived sequences in the sample based on sequence identity of the hybridization product to the nucleotide sequences of known bacterially-derived sequences.

According to embodiments of the present invention, there is provided a kit comprising any one of the bacterial sequence capture platforms described herein and instructions for using the platform.

In some embodiments, the kit further comprises a sample, wherein the platform is used for the detection, identification, and/or differentiation of bacterially-derived sequences in the sample.

In some embodiments, the sample is a biological sample or an environmental sample.

In some embodiments, the sample is a liquid sample or an aqueous sample.

In some embodiments, the sample is selected from the group consisting of a water sample, wastewater, sewage, greywater, blackwater, freshwater, liquid waste, seawater, drinking water, air, a gaseous sample, soil, a food sample, culture medium, and a swab of an inanimate surface or object.

In some embodiments, the sample is a wastewater sample or a sewage sample.

In some embodiments, the sample is a wastewater sample.

In some embodiments, the sample is a sewage sample.

In some embodiments, the sample is obtained from a sewage system, a drainage system, a plumbing system, or a water treatment facility.

In some embodiments, the sample is selected from the group consisting of saliva, mucus, a nasopharyngeal swab, serum, plasma, blood, urine, feces, cerebrospinal fluid, a bodily fluid, cultured cells, an organ tissue, and biopsied tissue.

In some embodiments, the sample comprises nucleic acids. In some embodiments, the nucleic acids in the sample are purified, enriched, and/or isolated. The platform of the kit may then be applied to the nucleic acids derived from the sample for the detection, identification, and/or characterization of vertebrate-infecting viruses in the sample.

According to embodiments of the present invention, there is provided a method for designing and/or constructing a database of probe sequences or a probe set comprising oligonucleotide probes for the detection, identification, and/or differentiation of bacteria and/or one or more of 16S ribosomal RNA, pathogenicity elements and/or AMR genes, comprising:
a) obtaining
   i) one or more species-specific or clade-specific marker gene sequences; or
   ii) one or more 16S ribosomal RNA sequences; or
   iii) one or more virulence factor sequences; or
   iv) one or more AMR gene sequences; or
   v) any combination of (i), (ii), (iii), and (iv); and
b) breaking the sequences obtained in step a. into fragments, wherein the fragments are the basis of the probes and are designed to be of a length, and spaced at a distance across the target sequences, such that the total number of probe sequences in the database or probes in the probe set corresponds to a desired range or number.

In some embodiments, the species-specific or clade-specific gene sequences are obtained from Metaphlan4 database.

In some embodiments, the 16S ribosomal RNA sequences are obtained from the SILVA database.

In some embodiments, the virulence factor sequences are obtained from the Virulence Factor Database (VFDB).

In some embodiments, the AMR genes are obtained from the Comprehensive Antibiotic Resistance Database (CARD).

In some embodiments, the desired range or number is less than one million.

In some embodiments, the method comprises a further step of synthesizing one or more of the oligonucleotide probes for which the sequence information was obtained in step b.

In some embodiments, the oligonucleotide probes are chosen from the group consisting of DNA, RNA, Bridged Nucleic Acids, Locked Nucleic Acids, and Peptide Nucleic Acids.

In some embodiments, the one or more oligonucleotide probes are synthesized on a cleavable microarray.

In some embodiments, the oligonucleotides are modified to comprise a composition for binding to a solid support, chosen from the group consisting of biotin, digoxygenin, ligands, small organic molecules, small inorganic molecules, aptamers, antigens, antibodies, and substrates.

According to embodiments of the present invention, there is provided a database of probe sequences for the detection, identification, and/or differentiation of bacteria and/or one or more of 16S ribosomal RNA, pathogenicity elements and AMR genes constructed by the method of constructing described herein and comprising one or more of sequence information, length, and origin of each oligonucleotide probe for which sequence information was obtained from the fragments in step b.

According to embodiments of the present invention, there is provided a probe set comprising oligonucleotides for the detection, identification, and/or differentiation of bacteria and/or one or both of pathogenicity elements and/or AMR genes, constructed by the method of constructing described herein.

In some embodiments, the probe set comprises approximately less than one million oligonucleotides.

According to embodiments of the present invention, there is provided a method for the detection, identification, and/or differentiation of bacteria and/or one or more of 16S ribosomal RNA, pathogenicity elements and/or AMR genes in a sample, comprising:
a) isolating nucleic acid from the sample;
b) contacting the nucleic acid or derivatives thereof with oligonucleotide probes of any one of the probe sets described herein to form hybridization products; and
c) detecting hybridization products between the nucleic acids from the sample and the oligonucleotide probes.

In some embodiments, the sample is chosen from the group consisting of a biological sample, an environmental sample, and a food sample.

In some embodiments, the sample is from a human.

In some embodiments, the subject is selected from the group consisting of domestic vertebrate animals, wild vertebrate animal and invertebrate animals.

In some embodiments, the method further comprises amplifying and sequencing one or more of the hybridization products from step (c).

In some embodiments, the method further comprises comparing one or more sample-derived sequences from the hybridization products from step (c) to one or more sequences of known bacteria, AMR genes and/or pathogenicity elements.

According to embodiments of the present invention, there is provided a kit for the detection, identification, and/or differentiation of bacteria, and/or one or more of 16S ribosomal RNA, pathogenicity elements and/or AMR genes, comprising any one of the databases or probe sets described herein.

For the foregoing embodiments, each embodiment disclosed herein is contemplated as being applicable to each of the other disclosed embodiments.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Examples

### Example 1—Design of Probes from Sequence Databases for Detection and Differentiation of Bacteria, Pathogenicity Elements and Antibiotic Resistance

To identify bacteria and associated virulence and resistance markers by capture sequencing, 120 bp oligonucleotide probes matching species-specific genomic or plasmid-encoded regions of bacteria, AMR genes/elements, and virulence factors were generated. These regions included species-specific genomic marker sequences, 16s rRNA genes, and AMR and virulence-associated genes from genomic and plasmid sequences. The marker sequences are the unique interspersed regions within genomes of a particular bacterial species within its core genomic sequence. These are termed as clade-specific marker genes in Metaphlan4. In the initial design 1333 bacterial species that are reported to be medically important (Table 1) were included. The design also included AMR genes and virulence associated factors from CARD and VFDB databases. The 120-mer oligonucleotides probes were spaced with a 60 nt distance along the target sequences. The resulting probe sets were clustered at 96% to obtain a final set of 988,786 probes. See Table 2.

TABLE 2
Databases used in probe design

| Database | Genetic Targets | |
|---|---|---|
| Metaphlan4 (vOctober 2022) | 90,776 | (894 species) |
| SILVA 138.1 | 1,325 | (1325 species) |
| CARD v3.2.5 | 4,750 | (4750 genes) |
| VFDB (December 2022) | 4,334 | (4334 genes) |

### Example 2—in Silico Validation of Marker Sequences for Bacterial Identification—Marker Sequence Validation

As an example, to show the use of the selected species-specific marker sequences for identifying bacterial species, bacterial species belonging to the same genus were taken and BLAST analysis was performed.

GenBank Refseq sequences for all bacterial species in Table 3 were downloaded and used for BLASTN analysis (−max_target_seqs 3-max_hsps 3-evalue 0.1) against the selected marker sequences for all Helicobacter species; for example, Helicobacter pylori (155 specific marker sequences), Helicobacter heilmannii (200 specific marker sequences), Helicobacter felis (200 specific marker sequences). All the species in Table 3 were evaluated for uniqueness.

Table 4 shows the number and percentage of our marker sequences that gave a BLAST hit with each of the tested species. For example, of the 155 marker sequences for H. pylori all hit H. pylori strain MT5135: only one hit in addition to tested Helicobacter species, H. felis (Table 4). In all instances, marker sequences (98-100%) hit the RefSeq genome for the respective Helicobacter species to which they are assigned. The only exception was H. cineadi, which belongs to the H. cinaedi/caniola/magdeburgensis complex of closely related species. In this case 99% of markers showed a BLAST hit with H. magdeburgensis. Accordingly, positive signal can represent multiple species within the complex; thus, further downstream analysis will be required for species designation.

TABLE 3
Bacterial species selected for validation of targeted marker regions

Helicobacter bilis
Helicobacter canadensis
Helicobacter canis
Helicobacter cinaedi
Helicobacter felis
Helicobacter fennelliae
Helicobacter heilmannii
Helicobacter magdeburgensis
Helicobacter pullorum
Helicobacter pylori
Helicobacter winghamensis

TABLE 4
Results of BLASTN analysis for Species-specific regions of Helicobacter species

| | No. of | | | |
|---|---|---|---|---|
| | marker | | No. of | |
| Target bacteria | sequences | Refseq genome hit ^{a} | hits (%) | |
| Helicobacter pylori | 155 | Helicobacter pylori strain MT5135 | 155 | (100) |
| | | Helicobacter felis ATCC 49179 | 1 | (0.006) |
| Helicobacter heilmannii | 200 | Helicobacter heilmannii isolate ASB1 | 200 | (100) |
| | | Helicobacter felis ATCC 49179 | 33 | (16.5) |
| | | Helicobacter pylori strain MT5135 | 12 | (6) |
| | | Helicobacter canis strain CCUG 32756T | 4 | (2) |
| | | Helicobacter fennelliae strain NCTC11613 | 3 | (1.5) |
| | | Helicobacter cinaedi PAGU611 | 2 | (1) |
| | | Helicobacter magdeburgensis strain MIT 96 | 2 | (1) |
| | | Helicobacter canadensis isolate MGYG-HGUT | 1 | (0.5) |
| Helicobacter felis | 200 | Helicobacter felis ATCC 49179 | 200 | (100) |
| | | Helicobacter heilmannii isolate ASB1 | 17 | (8.5) |
| | | Helicobacter winghamensis strain 2015D | 7 | (3.5) |
| | | Helicobacter fennelliae strain NCTC11613 | 7 | (3.5) |
| | | Helicobacter magdeburgensis strain MIT 96 | 1 | (0.5) |
| | | Helicobacter cinaedi PAGU611 | 1 | (0.5) |
| | | Helicobacter canadensis isolate MGYG-HGUT | 1 | (0.5) |
| Helicobacter fennelliae | 200 | Helicobacter fennelliae strain NCTC11613 | 200 | (100) |
| | | Helicobacter bilis WiWa acLZQ | 17 | (8.5) |
| | | Helicobacter magdeburgensis strain MIT 96 | 14 | (7) |
| | | Helicobacter cinaedi PAGU611 | 12 | (6) |
| | | Helicobacter winghamensis strain 2015D | 7 | (3.5) |
| Helicobacter bilis | 200 | Helicobacter bilis WiWa acLZQ | 200 | (100) |
| | | Helicobacter cinaedi PAGU611 | 9 | (4.5) |
| | | Helicobacter magdeburgensis strain MIT 96 | 7 | (3.5) |
| | | Helicobacter pullorum strain NCTC13156 | 6 | (3) |
| | | Helicobacter fennelliae strain NCTC11613 | 5 | (2.5) |
| | | Helicobacter canis strain CCUG 32756T | 5 | (2.5) |
| | | Helicobacter canadensis isolate MGYG-HGUT | 3 | (1.5) |
| | | Helicobacter winghamensis strain 2015D | 2 | (1) |
| Helicobacter canis | 200 | Helicobacter canis strain CCUG 32756T | 200 | (100) |
| | | Helicobacter magdeburgensis strain MIT 96 | 4 | (2) |
| | | Helicobacter cinaedi PAGU611 | 4 | (2) |
| | | Helicobacter fennelliae strain NCTC11613 | 3 | (1.5) |
| | | Helicobacter bilis WiWa acLZQ | 3 | (1.5) |
| | | Helicobacter winghamensis strain 2015D | 2 | (1) |
| Helicobacter | 200 | Helicobacter winghamensis strain 2015D | 200 | (100) |
| winghamensis | | | | |
| | | Helicobacter fennelliae strain NCTC11613 | 32 | (16) |
| | | Helicobacter canadensis isolate MGYG-HGUT | 19 | (9.5) |
| | | Helicobacter magdeburgensis strain MIT 96 | 9 | (4.5) |
| | | Helicobacter cinaedi PAGU611 | 8 | (4) |
| | | Helicobacter bilis WiWa acLZQ | 2 | (1) |
| Helicobacter | 200 | Helicobacter canadensis isolate MGYG-HGUT- | 200 | (100) |
| canadensis | | 01348 | | |
| | | Helicobacter fennelliae strain NCTC11613 | 33 | (16.5) |
| | | Helicobacter winghamensis strain 2015D | 15 | (7.5) |
| | | Helicobacter magdeburgensis strain MIT 96 | 1 | (0.5) |
| | | Helicobacter bilis WiWa acLZQ | 1 | (0.5) |
| Helicobacter pullorum | 200 | Helicobacter pullorum strain NCTC13156 | 200 | (100) |
| | | Helicobacter canadensis isolate MGYG-HGUT | 86 | (43) |
| | | Helicobacter winghamensis strain 2015D-0170 | 39 | (19.5) |
| | | chromosome | | |
| | | Helicobacter magdeburgensis strain MIT 96 | 5 | (2.5) |
| | | Helicobacter cinaedi PAGU611 | 3 | (1.5) |
| | | Helicobacter canis strain CCUG 32756T | 3 | (1.5) |
| | | Helicobacter bilis WiWa acLZQ | 3 | (1.5) |
| | | Helicobacter heilmannii isolate ASB1 | 1 | (0.5) |
| Helicobacter | 200 | Helicobacter magdeburgensis strain MIT 96 | 200 | (100) |
| magdeburgensis | | | | |
| | | Helicobacter cinaedi ^{b} PAGU611 | 198 | (99) |
| | | Helicobacter fennelliae strain NCTC11613 | 4 | (2) |
| | | Helicobacter winghamensis strain 2015D | 4 | (2) |
| | | Helicobacter bilis WiWa acLZQ | 3 | (1.5) |
| | | Helicobacter canis strain CCUG 32756T | 1 | (0.5) |

^{a} Only species with one or more BLAST hit are listed.
^{b} Helicobacter cinaedi is a member of larger Helicobacter cinaedi/caniola/magdeburgenesis complex

### Example 3—in Silico Validation of Marker Sequences for Bacterial Identification—Probe Set Validation

To validate the selected probes, multiple and single sequence alignments were performed and the number of probes that aligned to Refseq genomes of the genus Helicobacter as well as the specific contributions of each marker region were recorded. Of the total ˜0.99M probes, 11,196 probes mapped to species of the genus Helicobacter. These probes ranged in specificity from 89-100% for their designated species. In the 750-probe set designed for the H. cinaedi/caniola/magdeburgensis complex, 168 and 561 probes mapped to H. cinaedi and H. magdeburgensis, respectively. For H. pylori, additional probes from a virulence factor database (VFDB) that target specific virulence markers of this bacterium were designed. In summary, the analysis revealed discrete discriminatory alignment of probes which leads to efficient species level identification even within closely related genomes of same genus such as helicobacter.

TABLE 5
Results of clustering analysis from multiple and single sequence analysis

| | | | Probes | | | |
|---|---|---|---|---|---|---|
| | | | derived | Unique | | % |
| | Probes | | from | marker | Total | probes |
| | for genus | Total | marker | probes | mapped | specific |
| Refseq genomes | (mapped) | probes | Sequences | mapped | probes | target |
| Helicobacter pylori strain | 11,196 | 988,786 | 535 | 480 | 1185^{c} | 89.71 |
| MT5135 | | | | | | |
| Helicobacter heilmannii isolate | 11,196 | 988,786 | 1587 | 1499 | 1502 | 94.45 |
| ASB1 | | | | | | |
| Helicobacter felis ATCC 49179 | 11,196 | 988,786 | 1349 | 1329 | 1330 | 98.52 |
| Helicobacter fennelliae strain | 11,196 | 988,786 | 1461 | 1459 | 1464 | 99.86 |
| NCTC11613 | | | | | | |
| Helicobacter bilis WiWa acLZQ | 11,196 | 988,786 | 867 | 849 | 849 | 97.92 |
| Helicobacter canis strain CCUG | 11,196 | 988,786 | 863 | 840 | 840 | 97.33 |
| 32756T | | | | | | |
| Helicobacter winghamensis | 11,196 | 988,786 | 846 | 841 | 843 | 99.41 |
| strain 2015D | | | | | | |
| Helicobacter canadensis isolate | 11,196 | 988,786 | 1193 | 1193 | 1211 | 100 |
| MGYG-HGUT | | | | | | |
| Helicobacter pullorum strain | 11,196 | 988,786 | 1299 | 1299 | 1305 | 100 |
| NCTC13156 | | | | | | |
| Helicobacter magdeburgensis | 11,196 | 988,786 | 750 | 561 | 721 | 74.80 |
| strain MIT 96 | | | | | | |
| Helicobacter cinaedi PAGU611 | 11,196 | 988,786 | 750 | 169 | 669 | 22.53 |

^{c}additional probes mapped are majorly related to specific virulence factors of H. pylori from VFDB

### REFERENCES

Howell and Davis. 2017. Management of sepsis and septic shock. JAMA 317:847-848.
Rhee et al. 2017. Incidence and trends of sepsis in US hospitals using clinical vs claims data, 2009-2014. JAMA 318:1241-1249.
Aitor Blanco-Miguez et al. (2022) “Extending and improving metagenomic taxonomic profiling with uncharacterized with MetaPhlAn 4”, bioRxiv species preprint doi.org/10.1101/2022.08.22.504593.
Alcock B P et al. “CARD 2023: expanded curation, support for machine learning, and resistome prediction at the Comprehensive Antibiotic Resistance Database.” Nucleic Acids Res. 2023 Jan. 6; 51 (D1): D690-D699.
Liu B et al. “VFDB 2022: a general classification scheme for bacterial virulence factors.” Nucleic Acids Res. 2022 Jan. 7; 50 (D1): D912-D917.

1. A bacterial sequence capture platform for the detection, identification, and/or differentiation of bacterially-derived sequences in a sample,
the platform comprising a plurality of oligonucleotide probes, wherein the plurality comprises at least one oligonucleotide probe which comprises a hybridization portion partially or fully complementary to a portion of a bacterially-derived sequence selected from the group consisting of a bacterial gene sequence, a 16S ribosomal RNA sequence, a pathogenicity element sequence, a virulence factor sequence, and an antimicrobial resistance (AMR) gene sequence,
wherein the sequences of the hybridization portions of the oligonucleotide probes cluster at about 90-100% sequence identity,
wherein each hybridization portion of an oligonucleotide probe is about 5-300 nucleotides in length,
wherein different hybridization portions that each bind a different portion of the same bacterially-derived sequence are tiled across said bacterially-derived sequence and have an inter-probe spacing of about 20-100 nucleotides, and
wherein the plurality of oligonucleotide probes of the platform comprises 100,000 to 1,000,000 oligonucleotide probes, preferably less than about 1,000,000 oligonucleotide probes.

2. The platform of claim 1, wherein each hybridization portion of an oligonucleotide probe is about 50-200 nucleotides in length, preferably about 100-150 nucleotides in length, more preferably about 120 nucleotides in length.

3. The platform of claim 1, wherein the average length of the plurality of hybridization portions of oligonucleotide probes is about 120 nucleotides.

4. The platform of claim 1, wherein different hybridization portions that each bind a different portion of the same bacterially-derived sequence are tiled across said bacterially-derived sequence and have an inter-probe spacing of about 60 nucleotides.

1. The platform of claim 1, wherein the sequences of the hybridization portions of the oligonucleotide probes cluster at about 90%, 91%, 92%, 93%, 94%, 95%, 96%, 97%, 98%, 99%, or 100% sequence identity.

2. The platform of claim 1, wherein the plurality of oligonucleotide probes comprises hybridization portions partially or fully complementary to portions of bacterially-derived sequences comprising one or more bacterial gene sequences, one or more 16S ribosomal RNA sequences, one or more pathogenicity element sequences, one or more virulence factor sequences, and/or one or more antimicrobial resistance (AMR) gene sequences.

3. The platform of claim 1, wherein the bacterial gene sequence is a species-specific or clade-specific gene sequence.

4. The platform of claim 1, wherein each bacterially-derived sequence comprises a portion that is about 50-300 nucleotides in length and is partially or fully complementary to a hybridization portion of an oligonucleotide probe.

5. The platform of claim 1, wherein each hybridization portion is at least 90%, 91%, 92%, 93%, 94%, 95%, 96%, 97%, 98%, 99% or 100% complementary to a portion of a bacterially-derived sequence.

6. The platform of claim 1, wherein the plurality of oligonucleotide probes comprises at least one oligonucleotide probe which comprises a hybridization portion partially or fully complementary to a portion of a bacterially-derived sequence from a bacterial species listed in Table 1.

7. A method of screening a sample for bacterially-derived sequences, the method comprising:
a) exposing the sample, or nucleic acids isolated, amplified, and/or enriched from the sample, to the bacterial sequence capture platform of claim 1 to form one or more hybridization products, wherein each hybridization product comprises a nucleic acid of the sample and an oligonucleotide probe of the platform;
b) capturing the one or more hybridization products; and
c) identifying the presence of one or more bacterially-derived sequences in the sample based on the sequences of the one or more captured hybridization products;
thereby screening the sample for bacterially-derived sequences.

8. The method of claim 11, wherein nucleic acids in the sample are isolated and/or enriched prior to the exposing in step (a).

9. The method of claim 11, wherein the sample is a biological sample or an environmental sample.

10. The method of claim 11, wherein the sample is selected from the group consisting of saliva, mucus, a nasopharyngeal swab, serum, plasma, blood, urine, feces, cerebrospinal fluid, a bodily fluid, cultured cells, an organ tissue, and biopsied tissue.

11. The method of claim 11, wherein the sample is selected from the group consisting of an aqueous sample, a liquid sample, water, wastewater, sewage, greywater, blackwater, freshwater, liquid waste, seawater, drinking water, air, a gaseous sample, soil, a food sample, culture medium, and a swab of an inanimate surface or object.

12. The method of claim 11, wherein the sample is obtained from a human subject.

13. The method of claim 11, the method further comprising:
sequencing one or more detected hybridization products;
comparing the nucleotide sequence of the one or more hybridization products to nucleotide sequences of known bacterially-derived sequences; and
identifying and/or differentiating one or more bacterially-derived sequences in the sample based on sequence identity of the hybridization product to the nucleotide sequences of known bacterially-derived sequences.

14. A kit comprising the bacterial sequence capture platform of claim 1 and instructions for using the platform.

15. The kit of claim 18, further comprising a sample, wherein the platform is used for the detection, identification, and/or differentiation of bacterially-derived sequences in the sample.

16. The kit of claim 19, wherein the sample is a biological sample or an environmental sample.
